(19) United States
(12) Patent Application Publication
(10) Pub. No.: US 2011/0313009 A1
(43) Pub. Date: Dec. 22, 2011

(54) NSAID DOSE UNIT FORMULATIONS WITH H2-RECEPTOR ANTAGONISTS AND METHODS OF USE

(75) Inventors: George Tidmarsh, Portola Valley, CA (US); Leah Appel, Bend, OR (US)

(73) Assignee: Horizon Pharma USA, Inc.

(21) Appl. No.: 13/054,456

(22) PCT Filed: Jul. 17, 2009
(86) PCT No.: PCT/US09/51051

§ 371 Date: Jul. 8, 2011

Related U.S. Application Data
(60) Provisional application No. 61/081,611, filed on Jul. 17, 2008.

(51) Int. Cl. A61K 31/426; A61P 29/00

(52) U.S. Cl. 514/370

(57) ABSTRACT

The present invention generally relates to unit dosage forms of naproxen and H₂-receptor antagonists, comprising an immediate-release formulation of naproxen; an immediate-release formulation of an H₂-receptor antagonist, and a delayed-burst release formulation of an H₂-receptor antagonist.

This application claims the benefit of U.S. Provisional App. No. 61/081,611, filed Jul. 17, 2008, incorporated by reference in its entirety.

### FIELD OF THE INVENTION

The present invention provides pharmaceutical unit dosage forms comprising naproxen and H₂-receptor antagonists, and finds application in the fields of medicine and pharmacology.

### BACKGROUND OF THE INVENTION

Non-steroidal anti-inflammatory drugs (“NSAID(s)”) are known as effective analgesics for the treatment of mild to moderate pain. While generally regarded as safe, NSAIDs can cause gastritis, dyspepsia, and gastric and duodenal ulceration. Gastric and duodenal ulceration are a consequence of impaired mucosal integrity resulting from NSAID-mediated inhibition of prostaglandin synthesis. This side-effect is a particular problem for individuals who take NSAIDs for extended periods of time, such as patients suffering from rheumatoid arthritis or osteoarthritis.

The drug famotidine blocks the action of the histamine type-2 (H₂) receptor, leading to a reduction of acid secretion in the stomach. Reducing stomach acid by administering famotidine during treatment with certain NSAID drugs is reported to decrease incidence of gastrointestinal ulcers (see, e.g., Taha et al., 1996, “Famotidine for the prevention of gastric and duodenal ulcers caused by nonsteroidal anti-inflammatory drugs” N Engl J Med 334:1435-9, and Rostom et al., 2002, “Prevention of NSAID-induced gastrointestinal ulcers” Cochrane Database Syst Rev 4:CD002296).

### BRIEF SUMMARY OF THE INVENTION

In an embodiment, the invention provides a unit dosage form, comprising: (a) an immediate-release famotidine formulation comprising from about 10 mg to about 30 mg famotidine; (b) an immediate-release naproxen formulation, comprising from about 200 mg to about 600 mg naproxen; and (c) a delayed-burst release famotidine formulation, comprising from about 10 mg to about 80 mg (e.g., about 10 mg to about 30 mg) of famotidine, and a release-delaying agent.

Optionally, (a), (b) and/or (c) has a burst duration of about 60 minutes or less, for example about 30 minutes or less. For example, release from (a) and/or (b) occurs within about 30 minutes after administration. When desired, the release interval between (a) and (c) is at least about 0.5 hours. Optionally, the famotidine from (a) and/or (c) is effective to raise gastric pH above about 3.5 for at least about 4 hours.

When desired, the delayed-burst release of the famotidine from (c) extends the period during which gastric pH is raised. The raised pH can for example remain below about pH 6.5. Optionally, the release-delaying agent comprises an enteric material. The enteric material can be chosen if desired to allow release at or above a pH of about 6.5. In a variation, the immediate release famotidine can be adjusted to raise gastric pH to a desired range, and this can optionally be combined with a pH-sensitive delayed release of famotidine at a pH below this range. For example, the immediate release famotidine can cause the gastric pH to adjust to a pH above about 3.5 and below about 6.5, while the enteric material allows delayed drug release at or above a pH of about 6.5. In another example, the immediate release famotidine raises gastric pH above about 3.5 and below about 5.5, while the enteric material allows delayed drug release at or above a pH of about 5.5. Optionally, the enteric barrier comprises a material selected from the group consisting of cellulosic polymers, methacrylates, vinyl polymers and copolymers, and combinations thereof.

In other embodiments, the release-delaying agent is formulated for a fixed-time delayed release. For example, the release can be delayed by at least about 0.5 to 1 hour after administration to the patient. Optionally, one or more formulations within the unit dosage form comprises one or more pharmaceutically acceptable excipients selected from the group consisting of sugars, soluble salts, colorants, fillers, disintegrants, glidants, anti-lacking agents, anti-static agents, and any combination thereof.

The invention also provides therapeutic methods, for example, a method for treating or preventing pain or inflammation in a subject and/or of reducing the risk of an adverse side-effect of an NSAID, comprising administering a unit dosage form of the invention. The unit dosage form is optionally administered once, twice or thrice a day.

The invention also provides a method for manufacture of a unit dosage form, comprising (a) preparing an immediate release formulation comprising from about 200 mg to about 600 mg naproxen (e.g., in the form of naproxen sodium); (b) preparing an immediate release formulation comprising from about 10 mg to about 30 mg famotidine; (c) preparing a delayed-burst release formulation comprising from about 10 mg to about 80 mg (e.g., about 10 mg to about 30 mg) famotidine; and (d) combining the formulations of steps (a), (b) and (c), thereby resulting in a unit dosage form.

### SUMMARY OF FIGURES

FIG. 1. Exemplary unit dosage forms with immediate-release naproxen and immediate-release famotidine, and delayed-burst release famotidine (via osmotic rupture).

FIG. 2. Exemplary unit dosage forms with immediate-release naproxen and immediate-release famotidine, and delayed-burst release famotidine (via enteric-coated release).

FIG. 3. Exemplary unit dosage forms with immediate-release naproxen and immediate-release famotidine, and delayed-burst release famotidine (via erodible coating).

FIG. 4. Schematics of exemplary unit dosage forms. (A) osmotic rupturing multiparticulates, (B) osmotic rupturing tablet, (C) enteric coated multiparticulate, (D) erodible coated tablet, (E) osmotic rupturing multiparticulate with an immediate release blend in capsule, (F) osmotic rupturing tablet in an immediate release tablet, (G) osmotic rupturing multiparticulates in an immediate release tablet, (H) enteric coated multiparticulates with immediate release blend in capsule, (I) coated tablet in an immediate release tablet, (J) osmotic rupturing tablet with an immediate release blend in a capsule.

FIG. 5. Exemplary pharmaco-kinetic profile for delayed-burst release at four hours.

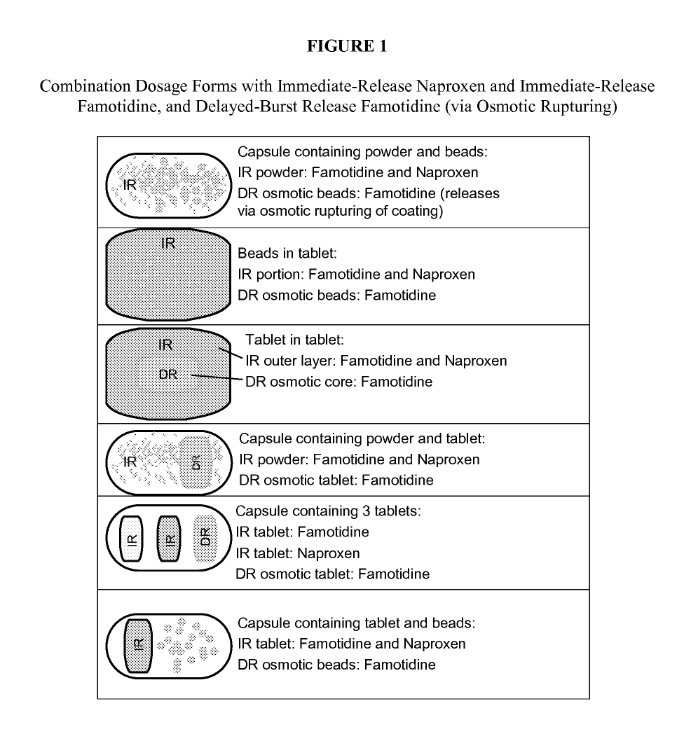

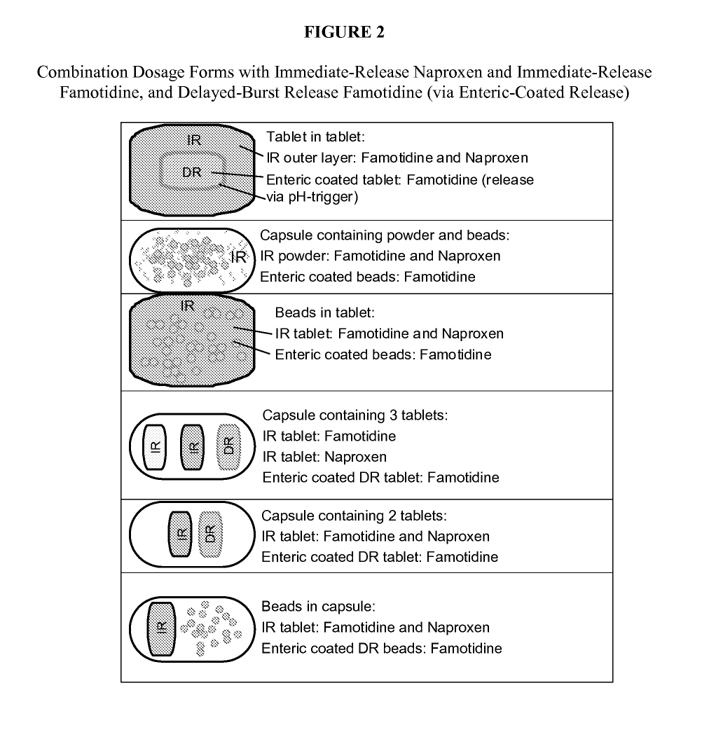

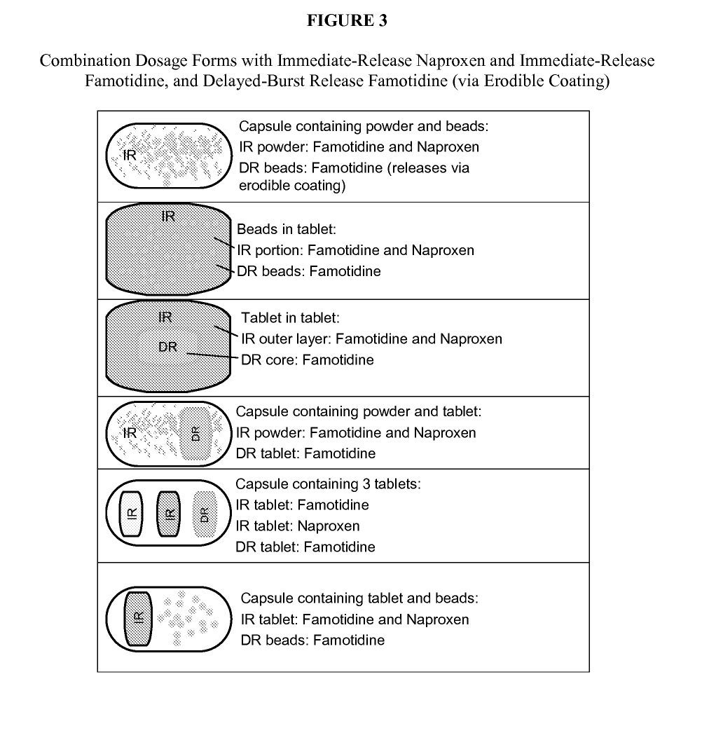

### DETAILED DESCRIPTION OF THE INVENTION

### I. Definitions

Unless stated otherwise, the following terms and phrases as used herein are intended to have the following meanings:

The terms “treatment of” and “treating” refer to taking steps to obtain beneficial or desired results, including clinical results. For purposes of this invention, beneficial or desired clinical results include, but are not limited to, alleviation or amelioration of one or more symptoms (e.g., pain, inflammation, ulceration, gastroesophageal reflux disease, etc.) of the disease or condition, diminishment of extent of the disease or condition, delay or slowing of the progression of the disease or condition, amelioration, palliation or stabilization of the disease or condition, and other beneficial results described below.

The term “risk” or “susceptibility” refers to a likelihood of an individual developing a disorder (e.g., a symptom, condition, illness, disorder or disease) relative to a control population. In one example, the control population can comprise one or more individuals in the general population (e.g., matched by age, gender, race and/or ethnicity, time since last meal, etc) who have not taken significant amounts of naproxen in the preceeding year and have not been diagnosed with a disorder related to increased gastric acid production.

The terms “oral dosage form,” “unit dose form,” and the like are used interchangably, and have their normal meaning in the art (i.e., refer to a pharmaceutical composition in the form of a tablet, capsule, caplet, gelcap, geltab, pill and the like).

The term “release” includes the provision (or presentation) of drug from a formulation to body tissues and/or fluids, such as those within the gastrointestinal tract. Generally, release refers to the release of significant amounts of drug. For instance, although in some instances a small portion of the drug may be released prematurely, this is generally less than 10% wt. (typically less than 5% or less than 1%) of the total drug in the formulation. In an embodiment, release is gauged by monitoring the concentration of drug in blood, serum or plasma. In another embodiment, release is monitored through the therapeutic effect produced by the drug. For example, the release of famotidine can be measured by monitoring gastric pH. Gastric pH can be monitored for example by using a pH electrode or probe such as a four-channel or two-channel probe. A non-invasive method of gastric pH measurement is set forth in U.S. Publication No. 20030129131. Alternatively, release can be gauged from an in vitro model such as an in vitro dissolution profile in an appropriate bio-relevant media such as phosphate buffer or simulated gastric fluid. It is understood that the exact time of release can vary within a unit dosage form. Thus, when release is stated herein to occur at a specified time, then it can be understood that the time specified is an average time of release.

“Famotidine” is 3-[2-(diaminomethyleneamino)thiazol-4-ylmethylthio]-N-sulfamoylpropionamidine, including polymorphic forms such as those designated Form A and Form B (see, e.g., U.S. Pat. Nos. 5,128,477 and 5,120,850) and their mixtures, as well as pharmaceutically acceptable salts thereof. Famotidine can be prepared using art-known methods, such as the method described in U.S. Pat. No. 4,283,408. Famotidine's properties have been described in the medical literature (see, e.g., Echizen et al., 1991, Clin Pharmacokinet. 21:178-94).

Naproxen is (+)-(S)-2-(6-methoxynaphthalen-2-yl) propanoic acid. Naproxen has analgesic, antiphlogistic and antipyretic properties, and is used for treatment of inflammatory diseases (e.g., arthritis) and to reduce pain (e.g. headaches, migraines, toothaches, back aches, muscle pain, post-operative pain and the like). Methods of making naproxen are well known in the art. As noted below, naproxen may be administered in various forms, including the free base, or a salt such as the sodium salt, or any combination of such forms. Reference herein to specific amounts of naproxen refer to the free base, and can be adjusted proportionally when other forms are used.

Reference to any of the compounds of the invention, e.g., famotidine, naproxen, or other H₂-receptor antagonists, also includes a reference to a physiologically acceptable salt thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of an hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds of the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived from a physiologically acceptable acid or base, are within the scope of the present invention.

For any measurement or value relating to the invention, such as risk, effect, gastric pH, increase in gastric pH, burst duration, release interval, amount of drug within a formulation, duration of release, concentration, etc, the value can be calculated as an average, for instance, the arithmetic or geometric mean or a median value, of measurements observed in a plurality of instances or events. For example, the release interval can be calculated as an average of release intervals measured in a plurality of subjects to whom the unit dosage forms of the invention are administered. The subjects can be for instance matched by age, weight, meal size, time since the last meal, etc. Alternatively, the release interval can be measured using in vitro assays.

When tradenames are used herein, applicants intend to independently include the tradename product and the active pharmaceutical ingredient(s) of the tradename product.

### II. Introduction

The present invention relates, inter alia, to pharmaceutical unit dosage forms comprising naproxen, an NSAID, and famotidine, an H₂-receptor antagonist. The unit dosage form comprises naproxen formulated for immediate release; famotidine formulated for immediate release; and famotidine formulated for delayed-burst release. Administering the H₂-receptor antagonist in both an immediate and delayed burst raises gastric pH and allows the delivery of the NSAID with reduced likelihood of NSAID-induced adverse effects such as ulceration.

### Amounts of Naproxen

The unit dose form contains a therapeutically effective amount of naproxen, in an amount in the range from about 150 mg to 700 mg, more often 200 mg to about 600 mg, for example about 250, 275, 300, 325, 350, 375, 400, 450, 500, 550, 600, 650 or 700 mg, of naproxen sodium. As recognized by those skilled in the art, when another form (e.g., naproxen or another salt of naproxen) is used in the formulation, the weight can be adjusted to provide an equivalent amount of naproxen.

### Amounts of Famotidine

The invention allows delivery of a therapeutically effective amount of famotidine or its pharmaceutically acceptable salts. A therapeutically effective amount of famotidine is usually in the range of about 10 mg to about 80 mg. The unit dosage forms of the invention can comprise about 30 to about 60 mg, such as about 35 to about 45 mg, e.g. about 40 mg. Accordingly, the amount of immediate release famotidine is usually in the range of about 10 mg to about 30 mg, e.g., 15 mg to 25 mg, for instance about 15, 18, 20, 22, or 25 mg and the amount of delayed-burst release famotidine may be in the range of about 10 mg to about 30 mg, or higher, e.g., 15 mg to 25 mg, for instance about 15, 18, 20, 22, or 25 mg. In some embodiments the amount of famotidine in the immediate release and delayed burst compartments or formulations is equal or about equal. In an example, the unit dosage form contains 40 mg famotidine in which the immediate release and delayed-burst release famotidines each may comprise about 20 mg famotidine. Such a unit dosage form allows administration of 80 mg/day famotidine with two-times-per day (BID) administration. The famotidine content can for example be approximately equally divided between the immediate release and delayed-burst release portions. In a non-limiting example the total famotidine content of the unit dose form is about 40 mg.

In some embodiments, the amount of famotidine in the delayed burst formulation is not the same as the amount of immediate release famotidine. For example the amount of delayed burst famotidine is greater than the amount of immediate release famotidine. Drug uptake in the gut is affected by various factors, including gastrointestinal permeability, transit time, drug solubility at the absorption site, the total area available for absorption, consumption with or without food, and/or pathologic conditions. H₂-receptor antagonists may be more poorly absorbed as they move down the gastrointestinal tract (e.g., from the stomach to proximal small intestine to distal small intestine, to colon). Because the delayed burst is released further down the digestive tract than the immediate release famotidine, the quantity of famotidine may be increase to, for example, achieve absorption of approximately the same amount of famotidine. Thus the delayed-burst release famotidine component may comprise a greater amount of famotidine than the immediate release famotidine component. to compensate for any decrease in gastrointestinal permeability as the drug moves down the GI tract. The delayed-burst release famotidine can, for example, be about 1.2-fold to 8-fold greater in amount than the immediate release famotidine, for example about 1.5-fold to two-fold greater, or about two-fold to about four-fold greater, or about four-fold to six-fold greater. The delayed-burst release famotidine can for example be about 1.2, 1.6, 2.0, 2.5, 3.0, 3.5 or 4-fold greater in amount than the amount of immediate release famotidine. Accordingly, the unit dose form can for example contain a delayed-burst release famotidine component of about 25 mgs, 30 mgs, 35 mgs, 40 mgs, 60 mgs or 80 mgs.

### Preferred Combinations of Famotidine and Naproxen—Amounts

In an embodiment, the unit dosage forms are designed to deliver a daily dose of about 400-1200 mg naproxen and about 80 mg famotidine with two times per day administration. For many applications the quantity of naproxen to be administered is about 550 mg naproxen sodium and the quantity of famotidine is about 40 mg (e.g., in the range 24 mg to 28 mg). This allows administration of 1100 mg/day naproxen sodium and 80 mg/day famotidine with BID administration.

In an embodiment, the immediate release and delayed-burst release famotidine each comprise from 10 to 80 mgs (e.g., 10 to 30 mgs) famotidine, and the unit dosage form also comprises about 200-600 mg naproxen formulated for immediate release. In an exemplary unit dose, for example, the immediate release and delayed-burst release famotidine each comprises about 20 mg famotidine, and the unit dosage form also comprises about 550 mg naproxen sodium.

In another embodiment, the unit dosage form comprises an immediate release famotidine component of about 20 mgs famotidine and an immediate release naproxen component of about 200-600 mgs naproxen, and a delayed-burst release famotidine component of about 40-80 mgs famotidine.

The immediate release famotidine component for example contains about 20, 25, 30 or 40 mgs famotidine. One exemplary unit dosage form comprises about 10-30 mgs immediate release famotidine and about 40-80 mgs delayed-burst release famotidine. Also for example, the unit dosage form can contain about 20 mgs famotidine and about 40 mgs famotidine. In another example the unit dosage form comprises about 15-25 mgs immediate release famotidine and about 60-80 mgs of delayed-burst release famotidine. Optionally the release interval is at least about 2-3 hours, e.g., about 4-6 hours.

### Quick Release of Drug (Burst)

As used herein, the rate of release of an API can be characterized by its “burst duration.” The “burst duration” is the time interval beginning from the time when release of an API from the dosage form begins, until the time when the API stops being released from the dosage form, generally because the dosage form is depleted of that particular API or drug formulation (end of burst). Generally, the present invention includes dosage forms that release active pharmaceutical ingredients (APIs) such as an NSAID and H₂-receptor antagonist (e.g., naproxen and famotidine) in relatively short bursts (or put differently, they are released with a short burst duration). The APIs within the unit dosage form generally have a burst duration of less than 120 minutes, e.g., less than 60 minutes, for instance less than about 30, 20, 15, 10, or 5 minutes. In this context, the immediate release and the delayed-release formulations are considered separate APIs (released in separate bursts), even if both contain the same drug, e.g., the drug is famotidine in both instances. For example, the immediate release famotidine burst begins and ends with the initiation and completion of release of immediate release famotidine from the unit dosage form; irrespective of whether famotidine from the delayed-burst release formulation has been released.

It will be appreciated that release (e.g., an immediate release or a delayed-burst release) is considered to begin upon release of a significant amount of the API in the unit dose form (e.g., at least 1%-2% or at least 5%, or sometimes at least 10% of the quantity of delayed burst famotidine) and does not include trivial or unmeasurable amounts. Optionally, release such as burst-type release can be considered to end at a timepoint after which any further amount of drug that is released is insignificant or low (e.g., less than 15%, often less than 10%) compared to amounts of drug released before this timepoint, even if residual amounts of the drug remain unreleased within the unit dosage form. Thus in estimating burst duration, the burst may be considered to end when at least 80%, more often at least 90%, or at least 95% of the famotidine in the delayed burst formulation is released. It will be apparent that the burst duration may be considered to be the length of time beginning after a certain amount of delayed burst famotidine (e,g, 5%) has been released and ending after a certain amount of delayed burst famotidine (e.g., 95%) has been released. In another embodiment, burst-type release can be considered to end at a timepoint after any further release occurs at an insignificant rate or a rate that is lower than the rate of release prior to the timepoint. In yet another embodiment, burst-type release can be considered to end when a lower, steady rate of release is achieved after an initial peaked release (burst). Thus the invention is not limited to embodiments in which essentially all (e.g., at least 95%, at least about 98% or at least about 99%) of the drug is released in a quick burst from the unit dosage form. In some embodiments, a burst can include a quick release of significant amounts of drug e.g., at least about 40%, 50%, 60%, 70%, 80%, 90%, 95% or 99% of drug within the unit dosage form, followed by a slower release of the remaining amounts of drug.

### Immediate Release Formulations:

Significant release of NSAID and H₂-receptor antagonist (e.g., naproxen and famotidine) from the immediate release formulations occurs within a relatively short period after administration in vivo (or exposure to solvent in vitro in standard dissolution assays). In some embodiments, the relatively short period can be for example within about 0.1 to 2 hours, e.g., about 10, 15, 20, 30, 40, 60 or 120 minutes. In some embodiments, the immediate release formulations release a majority of the drug, e.g., at least about 50%, 60%, 70%, 80%, 90%, 95% or 99% of drug from within the unit dosage form within such a relatively short period after administration. For example, about 80% of the drug can be released within about 30 or 40 minutes after administration, as measured standard dissolution assays such as those described herein. In contrast, delayed-burst release formulations typically release a minority (e.g., less than about 10%, less than 20% or less than 40%) of drug within 1 hour after administration. In an embodiment, the rate of release of drug and/or the absorption of drug from an “immediate release” formulation is neither appreciably nor intentionally retarded by the formulation itself. Thus, immediate release formulations exclude formulations which are specifically adapted to provide for “modified”, “controlled”, “sustained”, “prolonged”, “extended”, “delayed” or “delayed-burst” release of drug. Immediate release can be achieved by formulation of the API in combination with a pharmaceutically acceptable excipient which does not significantly retard the rate of drug release and/or absorption. Examples of excipients are well known in the art and certain examples are provided below.

### Delayed-Burst Release Famotidine

In an aspect, the H₂-receptor antagonist (e.g., famotidine) formulated for delayed-burst release has zero or relatively low release of drug during a lag period after administration to the subject; and then achieves a rapid release (“burst”) of drug after the lag period ends. The lag period is typically in the range of about 1 to 9 hours, often in the range of about 1 to 6 hours, such as in the range of about 1 to 3 hours, or in the range of 1 to 2 hours, or in the range of about 2 to 5 hours, or 3 to 6 hours. Optionally, the lag is about 4 hours after administration. Useful lag periods for the purposes of the present invention are in the range of about 0.5-10 hours, e.g., about 2-8 hours, sometimes about 3-6 hours, or about 0.5-2 hours, or about 1-3 hours. In an example, the lag is generally between about 3 hours and 5 hours, with an average lag time from about 3.5 to about 4.5 hours. Also for example, the lag is generally found to be between about 4 hours and 6 hours, for example from about 4 to about 5 hours. In yet another example, the lag is between about 4 hours and about 6 hours, for example from about 4 to about 5 hours.

Although the delayed-burst release from the unit dose form commences at a later time point than immediate release, once release begins the release pattern of the drug is similar to the pattern of immediate release, described above. For example, a relatively short burst duration, e.g., less than 120 minutes, for example within about 90 minutes, optionally less than 60 minutes, for instance less than about 30, 20, 15, 10, or 5 minutes, may be characteristic of both immediate release and delayed-burst release. Also for example, delayed-burst release can occur in a substantially unimpeded and/or relatively rapid manner once release begins. Many methods are known in the art for providing delayed-burst release, such as by diffusion, swelling, osmotic bursting or erosion (e.g., based on the inherent dissolution of the agent and incorporated excipients); certain methods are described below.

### Coordination of Release

Generally, the immediate-release formulations of famotidine and naproxen achieve immediate release at approximately the same time. Accordingly, the immediate release formulations are typically released within about 60, 45, 30, 20, 10 or 5 minutes of each other. In one embodiment the immediate-release formulations of famotidine and naproxen are released essentially simultaneously.

The immediate-release and delayed-burst release formulations of famotidine or other H₂-receptor antagonist results in two sequential bursts of famotidine, the first burst occurring relatively soon after administration and the second burst coming later. The time period between the first immediate burst of famotidine and the subsequent delayed burst of famotidine can be referred to as the “release interval.” In unit dose forms of the invention, the release interval can generally be in the range of about 0.5 to 9 hours, often in the range of about 1 to 6 hours, such as about 2 to 5 hours or about 1 to 3 hours, or 1 to 2 hours. In an embodiment, the release interval can optionally be about 0.5, 1, 1.5, or 2 hours. If so desired, the release interval can be longer, such as about 3 or 4 hours. The release interval can be determined in vitro or in vivo. Although the plasma concentration of a drug can lag behind the actual time of release in the GI tract, the release interval can be approximately determined in vivo as the time interval between the Cₘₐₓ (i.e., the maximum plasma concentration) of famotidine achieved by the immediate release formulation and the Cₘₐₓ of famotidine achieved by the delayed-burst release formulation. Alternatively, the release interval can be monitored through the increased plasma concentration of famotidine caused by delayed-burst release following immediate release, compared to that achieved by only the immediate release of famotidine. In yet another instance, the release interval can also be approximately determined by separately administering each famotidine API in a unit dosage form that lacks the other famotidine API.

In one exemplary unit dosage form, the delayed-burst release begins after a lag that is from about 2 hours to about 3 hours, or from about 3 hours to about 4 hours, or from about 4 hours to about 5 hours, or from about 5 hours to about 6 hours. Optionally, the delayed-burst release is timed to occur at a time when the unit dosage form is most frequently found in the small intestine in fasting and/or fed subjects. The immediate release of famotidine can for example occur within about 1 hour after administration, for example within about 30 minutes or within about 15 minutes.

Release can also be assessed using in vitro dissolution assays. In an example of immediate release, at least 75% of the famotidine and at least 75% of the naproxen in the dosage form are released within 15 minutes when measured in a Type II dissolution apparatus (paddles) according to U.S. Pharmacopoeia XXIX (USP) at 37° C. in 50 mM potassium phosphate buffer, pH 7.2 at 50 rotations per minute. Dissolution rates may be determined using other known methods. Generally an in vitro dissolution assay is carried out by placing the famotidine-naproxen unit dosage form(s) (e.g., tablet(s)) in a known volume of dissolution medium in a container with a suitable stirring device. Samples of the medium are withdrawn at various times and analyzed for dissolved active substance to determine the rate of dissolution. Dissolution may be measured as described for naproxen in the USP or, alternatively, as described for famotidine in the USP. In one approach, the unit dose form (e.g., tablet) is placed into a vessel of a United States Pharmacopeia dissolution apparatus II (Paddles) containing 900 ml dissolution medium at 37° C. The paddle speed is 50 RPM. Independent measurements are made for at least three (3) tablets, e.g., 6 tablets. The dissolution medium can be a neutral dissolution medium such as 50 mM potassium phosphate buffer, pH 7.2 (“neutral conditions”) or an acidic medium such as 50 mM potassium (or sodium) phosphate (or acetate or citrate) buffer, at pH 4.5. Typically a unit dose form is added to the vessel and dissolution is started. At specified times, e.g., 10, 20, 30, 45 or 60 minutes, a portion (e.g., 2 ml) of medium is withdrawn and the amount of API in solution is determined using routine analytical methods (e.g., HPLC).

By way of example, immediate release and/or delayed release of drug from the unit dosage form can be monitored using Apparatus II (Paddles) as described in U.S. Pharmacopeia, where the dissolution is conducted by placing one tablet into each of six vessels containing 900 mL of release media with temperature at 37° C. and speed of 50 rpm. Optionally, the release media of 0.1N HCl (pH 1.2 or 4.5) is used for stage 1 for 2 hours, and 0.1M phosphate buffer (pH 7.4) is used for stage 2 at 15, 30, 45, 60, 90 and 120 minutes and assayed for drug content by HPLC. In one strategy one or more examples of the unit dosage form can be manufactured for measurement purposes only, wherein the immediate release and delayed-burst release components each contain a detectable substance (“marker”) such as a dye. The delayed-burst release and immediate release components can for example contain different markers.

### Enhanced Therapeutic Effects of Sequential Release

The unit dosage forms disclosed herein provide an enhanced therapeutic effect compared to administration of naproxen and famotidine in separated dose forms. First, the release of each drug in a “burst” can achieve a higher maximal drug concentration in the bloodstream than sustained-release formulations that release a similar amount of the drug over a longer period of time, and. or the combined effect of the immediate release and the delayed-burst release of famotidine can increase gastric pH over baseline to a greater extent and/or for a longer time than either release would achieve alone. The unit dosage forms provide significant advantages over otherwise similar dosage forms that provide only an immediate release of famotidine, including a greater decrease in the frequency or risk of an adverse side effect of naproxen administration, or an increased delay in the development of such a side-effect.

### III. Excipients and Mechanisms of Delayed-Burst Release

A. Excipients Generally

The APIs of the invention are formulated with one or more pharmaceutically acceptable excipients. An “excipient,” as used herein, has its normal meaning in the art and is any component of an oral dosage form that is not the drug itself. Excipients include binders, lubricants, diluents, fillers, thickening agents, disintegrants, plasticizers, coatings, barrier layer formulations, lubricants, release-delaying agents and other components. Excipients are known in the art (see Handbook of Pharmaceutical Excipients, Fifth Edition, edited by Rowe et al., McGraw Hill; also see Pifferi et al., 2005, “Quality and functionality of excipients” Farmaco. 54:1-14; and Zeleznik and Renak, Business Briefing: Pharmagenerics 2004). In certain embodiments, one or more formulations of the unit dosage form include excipients, including for example and without limitation, one or more binders (binding agents), thickening agents, surfactants, diluents, release-delaying agents, colorants, fillers, disintegrants, lubricants, plasticizers, glidants, anti-caking agents, anti-tacking agents, anti-static agents, osmogens, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. In an embodiment, one or more excipients comprise cellulose derivatives, cross-linked polymers, sugars, soluble salts.

Useful diluents, e.g., fillers, include, for example and without limitation, dicalcium phosphate, calcium diphosphate, calcium carbonate, calcium sulfate, lactose, cellulose, kaolin, mannitol, sodium chloride, starches, powdered sugar, colloidal silicon dioxide, titanium oxide, alumina, talc, colloidal silica, microcrystalline cellulose, silicified microcrystalline cellulose and combinations thereof. Fillers that can add bulk to tablets with minimal drug dosage to produce tablets of adequate size and weight include croscarmellose sodium NF/EP (e.g., Ac-Di-Sol); anhydrous lactose NF/EP (e.g., Pharmatose DCL 21); and/or povidone USP/EP. In an embodiment, the filler for the delayed-release formulation comprises croscarmellose sodium NF/EP (Ac-Di-Sol) and/or anhydrous lactose NF/EP (Pharmatose DCL 21). In another embodiment, the filler for the immediate release formulations comprises povidone USP/EP and/or croscarmellose sodium NF/EP (e.g., Ac-Di-Sol). In another embodiment, the filler for the immediate release formulations comprises microcrystalline cellulose and SMCC.

Useful binder materials include, for example and without limitation, starches (including corn starch and pregelatinized starch), gelatin, sugars (including sucrose, glucose, dextrose and lactose), polyethylene glycol, povidone, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, colloidal silicon dioxide NF/EP (e.g., Cab-O-Sil M5P), Silicified Microcrystalline Cellulose (SMCC), e.g., Silicified microcrystalline cellulose NF/EP (e.g., Prosolv SMCC 90), and 2% silicon dioxide, mixtures thereof, and the like), veegum, and combinations thereof. In an embodiment, the binder can comprise colloidal silicon dioxide NF/EP (e.g., Cab-O-Sil M5P) and/or silicified microcrystalline cellulose NF/EP (e.g., Prosolv SMCC 90).

Useful lubricants include, for example, magnesium stearate NF, calcium stearate, sodium stearyl fumarate, stearic acid, mixtures thereof, and the like. Optionally, the lubricant (if present) comprises magnesium stearate NF.

Useful disintegrants include for example starches, clays, celluloses, alginates, gums, crosslinked polymers, colloidal silicon dioxide, mixtures thereof, and the like. In certain embodiments, croscarmellose sodium may be used. Croscarmellose sodium is a cross linked polymer of carboxymethyl cellulose sodium. Cross linking makes it an insoluble, hydrophilic, highly absorbent material, resulting in swelling properties, and its fibrous nature gives it water wicking capabilities. Croscarmellose sodium may be used in oral pharmaceutical formulations as a disintegrant for capsules, tablets and granules, and may be used in both direct-compression and wet-granulation processes. In certain embodiments, concentrations of up to 10%-80% w/w of croscarmellose sodium may be used, e.g., about 6% croscarmellose sodium NF/EP (Ac-Di-Sol).

Useful glidants include colloidal silicon dioxide, talc, calcium silicate, magnesium silicate, celluloses, and starches.

Useful osmogens include croscarmelose sodium, sodium starch glycolate, sodium chloride, sucrose, lactose, mannitol, mixtures thereof, and the like.

Useful surfactants include pharmaceutically acceptable non-ionic, ionic and anionic surfactants. An example of a surfactant is sodium lauryl sulfate. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. If desired, flavoring, coloring and/or sweetening agents may be added as well.

Optionally, a thickening agent can be added to provide the tablet with an accurately timed disintegration behavior. The tablet optionally disintegrates at a rate which is sufficiently slow to permit it to be swallowed easily, but fast enough to give an excellent suspension in water within 60 seconds. The thickening agent can be for example talc USP/EP, a natural gum, such as guar gum or gum arabic, or a cellulose derivative such as microcrystalline cellulose NF/EP (e.g., Avicel PH102), methylcellulose, ethylcellulose or hydroxyethylcellulose. A useful thickening agent is hydroxypropyl methylcellulose, an adjuvant which is available in various viscosity grades. The contribution of the thickening agent to the viscosity should be low. Therefore, the viscosity of a 2% solution of the thickening agent in water, measured at 20° C., should be less than 50 centipoise (cps), preferably less than 10 cps and most preferably about 5 cps. Optionally, the thickening agent can comprise microcrystalline cellulose NF/EP (e.g., Avicel PH102) and/or talc USP/EP.

Similarly, useful plasticizers include: acetylated monoglycerides; these can be used as food additives; Alkyl citrates, used in food packagings, medical products, cosmetics and children toys; Triethyl citrate (TEC); Acetyl triethyl citrate (ATEC), higher boiling point and lower volatility than TEC; Tributyl citrate (TBC); Acetyl tributyl citrate (ATBC), compatible with PVC and vinyl chloride copolymers; Trioctyl citrate (TOC), also used for gums and controlled release medicines; Acetyl trioctyl citrate (ATOC), also used for printing ink; Trihexyl citrate (THC), compatible with PVC, also used for controlled release medicines; Acetyl trihexyl citrate (ATHC), compatible with PVC; Butyryl trihexyl citrate (BTHC, trihexyl o-butyryl citrate), compatible with PVC; Trimethyl citrate (TMC), compatible with PVC; alkyl sulphonic acid phenyl ester, polyethylene glycol (PEG) or any combination thereof. Optionally, the plasticizer can comprise triethyl citrate NF/EP.

B. Mechanisms of Delayed-Burst Release

Delayed-burst release can be effected by the use of one or more release-delaying agents. Any combination of release-delaying agents, including the ones described herein, can be used in the unit dosage forms. The release-delaying agent optionally acts to increase the lag period before release begins from a unit dose form, but does not significantly retard release after release begins. The length of the lag period before delayed-burst release occurs can by controlled using methods known to those of skill in the art, for instance by varying the choice, combination, form, shape and/or amount of release-delaying agent(s).

The release-delaying agent may comprise a material which initially prevents release, but at some point allows rapid and unimpeded release of the drug. In some instances the release-delaying agent forms a barrier, capsule, covering or coating for the API. The delayed-burst release formulations can be prepared, for example, by coating a drug or a drug-containing composition with one or more release-delaying agents. In other instances, the release-delaying agent can be intermixed with or in co-solution with the drug. For example, delayed-burst release by osmotic rupture can be achieved by a unit dosage form comprising one or more swelling agents that are contained in combination with the drug within a semipermeable coating. The increase in volume of the swelling agent upon exposure of the unit dosage form to bodily fluids causes the semipermeable coating to rupture. In such agents, both the swelling agent and the semipermeable coating can be considered to be release-delaying agents. Thus, delayed release can be achieved by a combination of release-delaying agents, where each release-delaying agent does not necessarily delay release by itself.

Delayed-burst release can be achieved by various processes such as dissolution, diffusion, erosion (e.g., based on the inherent dissolution of the agent and incorporated excipients), and/or rupture (e.g., by swelling). Common mechanisms include bulk erosion of polymers which restrict diffusion of the drug, or surface erosion, e.g., of layered medicaments, or rupture. Rupture can be osmotically controlled, for instance by swelling that results from the osmotic infusion of moisture. Rupture can also result from the reaction of effervescent agents, e.g., citric acid/sodium bicarbonate, with water or other fluids that penetrate into the unit dosage form. Release, including delayed-burst release, from a unit dosage form can be achieved by more than one mechanism. For example, release can occur for example by simultaneous swelling and diffusion, simultaneous diffusion and erosion, and simultaneous swelling, diffusion and erosion.

Methods of making delayed-burst release formulations are within ordinary skill. Examples are presented herein and can also be found in numerous publications, including U.S. Pat. Nos. 4,865,849, 4,871,549, 4,897,270, 5,017,381, 5,110,597, 5,260,068, 5,260,069, 5,387,421, 5,472,708, 5,508,040, 5,593,697, 5840329, 6500457, 6531152, 6555136, 6627223, 6632451 and 7048945, all incorporated by reference.

Alternatively, delayed-burst release can be initiated by a triggering signal such as a fluctuation in temperature, or an electromagnetic pulse. See, e.g., US Patent Publication Nos. 20016251365, 2006997863, 20036514481, 20060057737, 20060178655, 20060121486, and 20060100608, all incorporated by reference. In other technologies, delivery can be though microchip delivery devices such as those described in U.S. Pat. No. 579,898, US2006123861 and U.S. Pat. No. 0,121,486, all incorporated by reference.

Two common classes of release-delaying agents are “enteric” (allowing release within a specific milieu of the gastro-intestinal tract) and “fixed-time” (allowing release after a pre time period after administration, regardless of gastro-intestinal milieu), each of which is discussed in more detail below. Enteric release-delaying agents for instance allow release at certain pHs or in the presence of degradative enzymes that are characteristically present in specific locations of the GI tract where release is desired. Enteric and fixed-time formulations are discussed in more detail below. The unit dosage forms can comprise more than one release-delaying agent from any class, such as a combination of enteric and fixed-time release-delaying agents.

i) Fixed-Time Delayed Release

In another embodiment, the release-delaying agent allows the release of drug after a predetermined lag period after the composition is brought into contact with body fluids (“fixed-time delayed release”). Unlike enteric release, fixed-time release is not particularly affected by environmental pH or enzymes.

A large number of fixed-time release-delaying agents are known to those of ordinary skill in the art. Exemplary materials which are useful for making the time-release coating of the invention include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose, cellulose acetate, cellulose acetate butyrate and ethyl cellulose; and other materials known to those of ordinary skill in the art. Other film-forming materials that can be used for this purpose include poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer. Other materials which can be used in the time-release coating include Eudragit NE, RL and RS, hydroxypropylcellulose, microcrystalline cellulose (MCC, Avicel™ from FMC Corp.), poly(ethylene-vinyl acetate) (60:40) copolymer (EVAC from Aldrich Chemical Co.), 2-hydroxyethylmethacrylate (HEMA), MMA, and calcium pectinate can be included. Substances that are used as excipients within the pharmaceutical industry can also act as release-delaying agents.

Common types of fixed-time release dosage forms include erodible formulations, formulations that undergo osmotic rupture, or unit dosage form that use any combination of mechanisms for delayed-burst release.

a) Osmotic-Rupture Formulations

Fixed-time release-delaying agents can optionally achieve a delayed-burst release by osmotic rupture. Examples of such RDAs include swelling agents, osmogens, binders, lubricants, film formers, pore formers, coating polymers and/or plasticizers.

In an embodiment, osmotic rupture is achieved by a delayed-burst release component which comprises a coated unit dosage form that contains the drug and a swelling agent within the semipermeable coating (e.g., ethylcellulose). The coating weight (thickness) of the semipermeable coating can be selected to delay release by osmotic rupture for a desired period. To identify the correct coating weight for a particular delay, unit dosage forms with a range of coating weights can be tested via in vitro dissolution to determine the burst time. Based on these results, a coating weight that achieves the desired lag period would be selected. In addition, the amount and/or ratio of coating strength modifier (e.g., talc) in the coating can be adjusted as well. Other formulation variables that can also be adjusted to obtain the desired release by osmotic rupture include the amount of sweller layer and sweller and/or fillers in the formulation. In the case of rupturing tablets, the amount of sweller would be selected to achieve the target release, while still providing the tablet with sufficient compressibility and acceptably low friability to be manufacturable.

In an embodiment, the unit dosage form can comprise one or more “diffusion regulators” that control the permeation of bodily fluids into the drug-containing core. Exemplary diffusion regulators include hydrophilic polymers, electrolytes, proteins, peptides, amino acids and others known to those of ordinary skill in the pharmaceutical sciences. In an example, the fixed-time release-delaying agent comprises a coating that permits release of drug after a fixed period. The thickness of the coating can affect the time required for penetration of fluids into the formulation. For example and not limitation, a diffusion controlling time release coating that provides release after a fixed delay period of about 0.5-2.5 hours could be about 200-1000 microns thick, and one that provides a release after a fixed delay period of about 2.5-5.0 hours could be about 1000-3000 microns thick.

Examples of osmotic rupturing multiparticulates are demonstrated in the literature (See, e.g., Dashevsky, et al, International Journal of Pharmaceutics, 318, (2006) 124-131; Mohamed, et al, Drug Development and Industrial Pharmacy, 33 (2007) 113-119; U.S. Pat. No. 4,871,549; Ueda, S., et al, Journal of Drug Targeting, 2 (1994) 35-44; Ueda, S., et al, Chemical Pharmaceutical Bulletin, 42(2), (1994) 359-363; Ueda, S., et al, Chemical Pharmaceutical Bulletin, 42(2), (1994) 364-367). Examples of osmotic rupturing tablets are demonstrated in the literature (U.S. Pat. No. 4,871,549; Theeuwes, F., Journal of Pharmaceutical Sciences, Vol. 64, No. 12, (1975) 1987-1991; Sungthongjeen, S., et al, Journal of Controlled Release, 95, (2004) 147-159).

The sweller used in the sweller layer is generally a super-disintegrant (e.g., croscarmellose sodium, sodium starch glycolate, low-substituted HPC). The different super-disintegrants have different swelling energies; here the common super-disintegrants are listed in order of decreasing swelling energy: croscarmellose sodium>low-substituted HPC>sodium starch glycolate>crospovidone (See, e.g., Roy, P., et al., Current perspectives, Journal of Controlled Release, 134 (2009) 74-80). For a given formulation with the same level of disintegrant and the same coating weight, super-disintegrants with higher swelling energies will provide shorter burst times and shorter burst durations (See, e.g., Dashevsky, et al, International Journal of Pharmaceutics, 318, (2006) 124-131). Based on this, the super-disintegrant with the highest swelling energy was selected for this formulation. If other super-disintegrants were used, a higher level would be needed to achieve the same performance.

The level of sweller used in the formulation will impact the burst time and duration as well. Below a certain level of sweller in the formulation, burst duration will grow longer as the amount of sweller decreases. At a certain level, the burst duration will reach a minimum (See, e.g., Dashevsky, et al, International Journal of Pharmaceutics, 318, (2006) 124-131). This level will vary with other formulation parameters, but will generally be in the range of 20-50 wt % coating.

The in vitro dissolution test can be adapted to test osmotic release. For example, the dissolution medium can be a neutral dissolution medium such as 50 mM potassium phosphate buffer, pH 7.2 or an acidic medium such as 0.01N HCl, pH 2. The dissolution medium should be at physiologically relevant osmotic pressure (7 atm) for testing these types of formulations, since the difference in pressure between the media and the multiparticulate can impact performance. The osmotic pressure of the media can be adjusted by adding the appropriate level of salts (e.g., NaCl). Typically a unit dose form is added to the vessel and dissolution is started. At specified times, e.g., 30 minutes, 1, 2, 3, 4, 5, 6 hours, a portion (e.g., 10 mL) of medium is withdrawn and the amount of API in solution is determined using routine analytical methods (e.g., HPLC). Dissolution should be tested in both neutral and acidic media to design the formulation to have performance relatively insensitive to pH.

b) Erodible Formulations

Erodible formulations provide another example of fixed-time release formulations. The release delay from an erodible coated tablet can be adjusted by those of ordinary skill in the art by regulating the erodible layer coating weight. To identify the correct coating weight, tablets over a range of coating weights can be tested via in vitro dissolution (and/or erosion) to determine the burst time. Other formulation variables that may affect performance are the selection of the coating layer polymer type and viscosity. Examples of erodible coated tablets are demonstrated in the literature [Sangalli, M. E., et. al., Journal of Controlled Release, 73 (2001) 103-110; Gazzaniga, A., et. al., International Journal of Pharmaceutics, 108 (1994) 77-83].

In an embodiment, the unit dosage form can comprise one or more “erosion regulators” that control the erosion rate of the coating. Any material or combination of materials may serve as an erosion regulator. Exemplary erosion and/or diffusion regulators include hydrophilic polymers, electrolytes, proteins, peptides, amino acids and others known to those of ordinary skill in the pharmaceutical sciences. The thickness of the coating can affect the time required for erosion of the coating. For example and not limitation, an erodible time-release coating that provides release after a fixed period of about 0.5-2.5 hours could be about 100-2000 microns thick, and one that provides release after a fixed delay period of about 2.5-5.0 hours could be about 2000-5000 microns thick.

ii) Enteric Release

The release-delaying agent may comprise an “enteric” material that is designed to allow release upon exposure to a characteristic aspect of the gastrointestinal tract. In an embodiment, the enteric material is pH-sensitive and is affected by changes in pH encountered within the gastrointestinal tract (pH sensitive release). The enteric material typically remains insoluble at gastric pH, then allows for release of the active ingredient in the higher pH environment of the downstream gastrointestinal tract (e.g., often the duodenum, or sometimes the colon). In another embodiment, the enteric material comprises enzymatically degradable polymers that are degraded by bacterial enzymes present in the lower gastrointestinal tract, particularly in the colon. Optionally, the unit dosage form is formulated with a pH-sensitive enteric material designed to result in a release within about 0-2 hours when at or above a specific pH. In various embodiments, the specific pH can for example be about 4.5, 5, 5.5, 6, or 6.5. In particular embodiments, the pH-sensitive material allows release of at least 80% of the drug within 1 hour when exposed to a pH of about 5.5 or higher. In another embodiment, the pH-sensitive material allows release of at least 80% of the drug within 1 hour when exposed to a pH of about 6 or higher.

In formulations that allow for pH-sensitive release, the amount of immediate-release famotidine is preferably calibrated to raise gastric pH, but not to a level at which the pH-sensitive release-delaying agent would also allow delayed-burst release. In an aspect, the pH-sensitive release-delaying agent allows delayed-burst release of famotidine at or above a pH of about 5.0, 5.5, 6.0 or 6.5, and the immediate-release famotidine raises the gastric pH, but not up to a pH at which the release-delaying agent allows release. For instance, the gastric pH is raised by the H₂-receptor antagonist, but still remains below about 4.5, 5.0, 5.5, 6.0 or 6.5.

In some embodiments, a release-delaying agent is included with a desired amount of famotidine in the delayed-burst release formulation. For example, famotidine may be mixed with (e.g., blended, intermixed or in continuous phase with) and/or contained within (e.g., encapsulated within or coated with) one or more release delaying agents, for example as shown in Example 3-24. For example, the delayed-burst release formulation can be in the form of one or more capsules containing famotidine. In other instances, famotidine can be in multiparticulate form such as granules, microparticles (beads) or nanoparticles, coated with release-delaying agent, for example as shown in Examples 5-11.

Materials used for enteric release formulations, for example as coatings, are well known in the art and include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. A preferred coating agent is methacrylic acid copolymer NF (Eudragit L100-55). Combinations of different enteric materials may also be used. Multi-layer coatings using different polymers may also be applied. In some instances, the enteric materials causes a delay of drug release in the range of about 0.5 to about 6 hours, sometimes about 0.5 to about 4 hours. The properties, manufacture and design of enteric delivery systems are well known to those of ordinary skill in the art. See, e.g., Development of Biopharmaceutical Parenteral Dosage Forms (Drugs and the Pharmaceutical Sciences), by Bontempo (Publishers: Informa Healthcare (Jul. 25, 1997)).

Those of ordinary skill in the art can adjust the lag period before delayed-burst release from enteric coated multiparticulates by varying the enteric layer coating weight and composition. For example, where time in the stomach is <4 hours and some amount of protection (1-3 hours) is desired after the dosage form leaves the stomach, then an appropriate level of coating that provides up to 4 hours of protection between administration and drug release. can be prepared. To identify the correct coating weight, samples of multiparticulates would be pulled from the fluid bed coater over a range of coating weights and tested via in vitro dissolution to determine the appropriate coating level. Based on these results, the correct coating weight would be selected. An example of an enteric coated multiparticulate can be found in U.S. Pat. No. 6,627,223.

### IV. Unit Dosage Forms

### Monolithic Dosage Forms

Generally, the unit dosage form is monolithic in nature, e.g., in the form of a tablet or capsule or a caplet (capsule-form tablet), for example as shown in Examples 14-24. Monolithic unit dosage forms may vary in shape and may be, for example, round, ovoid, oblong, cylindrical (e.g., disk shaped) or any other geometric shape, for example rectilinear. For example, the unit dosage form can have a disk or ovoid shape, or a shape like a flattened disk or torpedo. The edges of can be beveled or rounded. In less preferred non-monolithic embodiments, the unit dosage form itself comprises more than a two or three separate subunits, e.g., two separate tablets, one designed for immediate release of famotidine and naproxen, and the second tablet designed for delayed-burst release of famotidine. The unit dosage form can be provided in certain embodiments (e.g., non-monolithic embodiments) as a kit comprising separate components. In an example, the delayed-burst release formulation is in monolithic form, for example as shown in Examples 9, 12 and 13. Optionally, the delayed-burst release formulation is for osmotic rupture, for example as shown in Examples 5-9.

### Multiparticulate Forms

Although the unit dosage form is generally a monolithic entity, APIs contained within the unit dosage form need not be in monolithic form. For instance, one or more APIs can be multiparticulate in form, for example as shown in Examples 1-13. APIs in multiparticulate form for example comprise a plurality of drug-containing beads, particles or granules. Such multiparticulate forms are for instance incorporated into a unit dosage form that is a tablet or capsule. In an aspect, one or more APIs (e.g., immediate release formulations) can be in the form of a powder blend, independently or together with tableted or capsular API(s). One or more APIs can also be in the form of a gel or sol.

### Tablet/Capsule Combinations of Formulations—Coated Core Tablets

One or more APIs can be present in the form of a tablet or capsule within the unit dosage form. APIs in tablet form can be incorporated into a unit dosage form that is a capsule. Alternatively, tablet-type APIs can be used as an inner core in a “coated core” tablet-type unit dosage form. In one such example, the unit dosage form comprises a multilayered tablet, with an inner core of famotidine APIs for delayed release, and one or more outer layers that comprise immediate-release famotidine and/or naproxen. One such embodiment, as described in the Examples, includes a unit dosage form comprising an enteric-coated famotidine tablet to provide the delayed burst release, contained within a larger tablet that contains the immediate release formulations of famotidine and naproxen.

### Tablet Delayed-Burst Release Famotidine in Capsule Dosage Form

In another example, the delayed-burst release famotidine APIs is in the form of a tablet or capsule, while the immediate release formulation(s) are in the form of a flowable powder. The APIs may be formulated independently or in combination. The final unit dosage form can be in the form of a capsule, and in such cases the delayed-burst release famotidine can be sized and shaped so as to be easily accommodated within the capsule, while allowing for inclusion of the immediate-release famotidine and/or naproxen as well. As understood by those skilled in the art, the tablet or capsule configuration of the delayed-burst release famotidine formulation may be specifically sized and shaped for such a purpose. Upon administration, the unit dosage form capsule releases the immediate release naproxen and/or famotidine flowable powder(s), and the delayed-burst release tablet or capsule releases famotidine at a later timepoint.

### Separability of Formulations

In an embodiment, the unit dosage form can include more than one discrete and separable formulation, wherein each formulation comprises at least one drug of the unit dosage form. In such instances, the formulations can be separately prepared, and then combined into the final unit dosage form. In another embodiment, drugs can be coformulated such that they are physically inseparable. In one exemplary unit dosage form, both the naproxen and famotidine immediate release formulations are powders, and the unit dosage form comprises a powder blend in which particles containing famotidine are intermixed with particles containing naproxen. In another embodiment, a co-solution of famotidine and naproxen is processed, e.g., by spray-drying or lyophilization, into an immediate release powder, wherein both famotidine and naproxen can be found together within a single particle of the powder. Optionally the famotidine and naproxen can be in solid solution within such particles, and/or form a single continuous phase.

### IV. Administration

The dosage ranges described above are preferred adult doses and may vary depending upon the age and weight of the patient as would be known by those skilled in the pharmaceutical arts. In an embodiment, the unit dosage forms contain naproxen and famotidine and are administered twice a day (BID). Accordingly the unit dosage forms are typically administered about every 12 hours.

Although administration twice a day is preferred, the unit dosage form can be administered more frequently (e.g., three or four times a day) or less frequently (e.g., once a day, or about once every few days). The unit dosage form can be administered at specific points in the day or schedule of a subject, e.g., morning, afternoon, evening, night, before or during or after meals, before bedtime, etc.

### V. Therapeutic and Prophylactic Methods

The invention also provides methods of treatment and/or prophylaxis of an adverse side-effect of NSAID administration, by co-administering naproxen with famotidine using the unit dosage forms of the invention. The adverse side-effect is for example acidity or the development of an ulcer. Accordingly, these methods are especially useful to treat a subject who is suffering from NSAID-induced ulcers or acidity, or is at elevated risk for developing such ulcers.

A subject is at elevated risk for developing an NSAID-induced ulcer if the subject is more susceptible than an average individual to development of an ulcer when under treatment with NSAID. A high odds ratio for risk of development of NSAID-associated ulcer complications is seen in individuals with a past complicated ulcer (odds ratio 13.5), individuals taking multiple NSAIDs or NSAIDs plus aspirin (odds ratio 9.0); individuals taking high doses of NSAIDs (odds ratio 7.0), individuals under anticoagulant therapy, such as low dose aspirin (odds ration 6.4), individuals with a past uncomplicated ulcer (odds ratio 6.1), and individuals older than 70 years (odds ratio 5.6) See, e.g., Gabriel et al., 1991, Ann Intern Med. 115:787; Garcia Rodriguez et al. 1994, Lancet 343:769; Silverstein et al. 1995, Ann Intern Med. 123:241; and Sorensen et al., 2000, Am J. Gastroenterol. 95:2218. In addition, subjects older than 80 years of age and/or subjects with a history of NSAID-associated serious gastrointestinal complications (perforation of ulcers, gastric outlet obstruction due to ulcers, gastrointestinal bleeding) are also generally at high risk for developing NSAID-induced ulcer.

In another embodiment the invention relates to therapeutic methods comprising administering a unit dosage form of the invention to the subject in need of treatment. One aspect of the invention provides for treatment or prophylaxis of any subject suffering from or at elevated risk for conditions suitable for treatment with an H₂-receptor antagonist. These include conditions in which the stomach produces excess gastric acid and/or conditions in which acid comes up into the esophagus, such as acidity, heartburn, ulceration, gastritis, gastroesophageal reflux disease (GERD) and/or esophagitis. In an example, a unit dosage form of the invention is administered to subjects suffering from (or at elevated risk of) gastric acidity and/or gastrointestinal ulcers such as gastric and/or duodenal ulcers.

### Elevation of Gastric pH

The famotidine is released from the immediate release and/or delayed-burst release formulation in sufficient amount to raise the gastric pH above about 3.5, for instance, e.g., above about 4.0, 4.5, 5.0, 5.5, 6.0 or greater. In an example, the gastric pH is raised above about 5.5. In an aspect, the immediate release and/or delayed-burst release formulations of the unit dosage form allow release of the famotidine in sufficient amount to raise the gastric pH levels above a pH in which NSAID-induced adverse side effects, e.g., ulceration or acidity, typically occurs. In some cases, the delayed-burst release of famotidine further increases the gastric pH by about 0.2, 0.5, 0.7, or 1.0 units above the already-increased pH achieved by the immediate release of famotidine from the immediate-release famotidine. Optionally, the total time for which gastric pH is raised following administration is about 1, 2, 4, 5, 6, 8 or 10 hours.

### Naproxen Therapy/Prophylaxis

The invention also provides for treatment or prophylaxis of any subject in need of NSAID (e.g., naproxen) treatment with the dosage forms of the invention. A “subject in need of NSAID treatment” is an individual who receives therapeutic benefit from administration of an NSAID, such as an individual suffering from a condition associated with pain and/or inflammation, such as mild to moderate pain, dysmenorrhea, inflammation, osteoarthritis, and many other conditions. In one embodiment, the subject in need of NSAID treatment is under treatment for a condition associated with chronic pains (e.g., chronic low back pain, chronic regional pain syndrome, chronic soft tissue pain), or a chronic inflammatory condition. Such conditions include arthritic pain (e.g., pain associated with osteoarthritis and rheumatoid arthritis), neuropathic pain, and post-operative pain, chronic lower back pain, cluster headaches, herpes neuralgia, phantom limb pain, central pain, dental pain, neuropathic pain, opioid-resistant pain, visceral pain, surgical pain, bone injury pain, pain during labor and delivery, pain resulting from burns (including sunburn), post partum pain, migraine, angina pain, and genitourinary tract-related pain including cystitis, the term also refers to nociceptive pain or nociception. In general, a subject under treatment for a chronic condition requires NSAID treatment for an extended period, such as at least one month, at least four months, at least six months, or at least one year.

In another embodiment, the subject in need of NSAID treatment is under treatment for a condition that is not chronic, such as acute pain, dysmenorrhea or acute inflammation.

### VI. Methods of Manufacture

The APIs may conveniently be presented in unit dosage form and may be prepared by any of the methods well known in the art of pharmacy. Techniques and formulations generally are found in REMINGTON'S PHARMACEUTICAL SCIENCES (Mack Publishing Co., Easton, Pa.). Such methods include the step of bringing into association the active ingredients of one or more with any additional excipients. In general the APIs are prepared by uniformly and intimately bringing into association the active ingredient with liquid carriers or finely divided solid carriers or both, and then, if necessary, shaping the product or filling capsules.

In certain preferred embodiments, the unit dosage form may be prepared in any pharmaceutically acceptable manner, such as by: (a) preparing an immediate release formulation comprising a therapeutically effective amount of naproxen; (b) preparing an immediate release formulation comprising a therapeutically effective amount of famotidine; (c) preparing a delayed-burst release formulation comprising a therapeutically effective amount of famotidine, and at least one release-delaying agent; and (d) combining the formulations within a unit dosage form. The immediate release famotidine and naproxen can be contained in the same single formulation and/or prepared together. In an aspect, the amount of famotidine is effective to raise gastric pH above about 3.5 over a predetermined period of time following administration.

### Methods of Making Tablets, Capsules

As mentioned, in an embodiment the unit dosage form and/or one or more formulations are in tablet form. Various methods of preparation of tablets are well known to one of ordinary skill in the art. See, e.g., Pharmaceutical Dosage Forms: Tablets, Third Edition, by Larry L. Augsburger and Stephen W. Hoag (publisher: Informa Healthcare; Dec. 15, 2007). These methods include direct compression and granulation (e.g., wet or dry or fluid-bed).

In yet other embodiments, the unit dosage form and/or one or more APIs is/are in capsule form. Diverse capsule manufacturing and design methods are well known to one of ordinary skill in the art. See, e.g., Pharmaceutical Preformulation and Formulation: A Practical Guide from Candidate Drug Selection to Commercial Dosage Form, by Mark Gibson (publishers: Informa Healthcare, Aug. 1, 2001). When the unit dose form is a capsule, the method further comprises preparing one or more APIs into a form for loading and/or delivery, e.g., as a tablet, capsule and/or powder, and loading the formulations into the capsule to form the pharmaceutical unit dose.

The amount of active ingredient included in the various formulations will vary depending upon the ingredient, the subject to be treated, and the particular disease or condition of interest, as generally recognized by those skilled in the art.

### VII. Examples

### Example 1

### Manufacture of Immediate Release Blend of Famotidine and Naproxen and a Delayed-Burst Release Tablet of Famotidine

An exemplary unit dosage form in accordance with certain embodiments of the invention was prepared as follows. In this example, the delayed-burst release formulation was in the form of an enterically-coated famotidine tablet, while the immediate-release formulations of famotidine and naproxen were prepared as flowable powders. The famotidine was blended and then coated with a delayed release enteric polymer coating. This enteric coated tablet was combined with the two immediate-release powders of famotidine and naproxen and compressed as a single tablet.

For the famotidine delayed-burst release core tablet, the following ingredients were charged, in the following order: lactose anhydrous, croscarmellose sodium, famotidine, colloidal silicon dioxide and microcrystalline cellulose into a V-blender and blended for 10 minutes. The blend was then passed through a 20 mesh screen and charged back into the V-blender and blended for an additional 10 minutes. The required amount of magnesium stearate (adjusted to the blend weight) was passed through 40 mesh screen and charged into the same blender containing the famotidine blend and the mixture was blended again for 3 minutes. After confirming the uniformity of the final blend, the final blend was compressed using a suitable tablet press to get famotidine core tablets. The core tablets were coated with an enteric/delayed release coating comprising Eudragit polymer in additional ingredients and solvent to get famotidine core delayed-burst release tablets.

The immediate-release blend of naproxen sodium and famotidine was prepared as follows. First, the naproxen sodium and famotidine was passed through a 30 mesh screen and mixed in a V-blender for 5 minutes. Microcrystalline cellulose, SMCC-90, Povidone, and croscarmellose sodium were charged through a 30 mesh screen into the V-blender and mixed for 7 minutes. Colloidal silicon dioxide and magnesium stearate were passed through a 40 mesh screen into the V-blender and mixed for 3 minutes, and final blend uniformity was checked.

### Example 2

### Manufacture of a Unit Dosage Formulation in Tablet Form Containing the Components of Example 1

To assemble the final unit dose formulation, 515 mg of the immediate-release famotidine/naproxen sodium blend was filled into a die, one famotidine core delayed-burst release tablet was inserted, and another 515 mg of the immediate-release famotidine/naproxen sodium blend was added into the die. The tablets were then compressed and in-process checks were performed. 40 tablets were packaged into a 100-cc bottle.

Table 1 below shows the preparation of the enteric coated famotidine delayed-burst release tablet. Table 2 shows the manufacture of the final unit dosage form. Table 3 shows the composition of an exemplary tablet.

TABLE 1
Flow Diagram of Compression of Famotidine Core Tablets

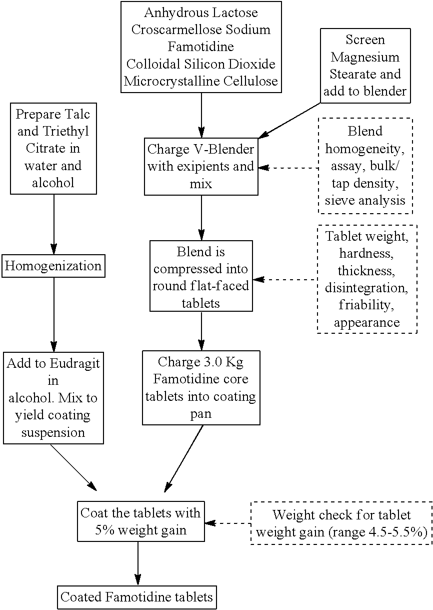

TABLE 2
Flow Diagram of Naproxen/Famotidine Tablets (HZT-602)

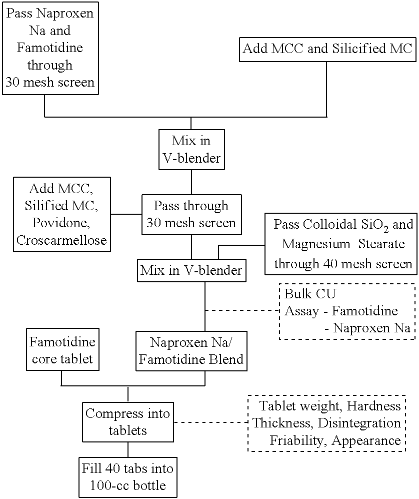

TABLE 3
Composition of an Exemplary Tablet

| | | | |
|---|---|---|---|
| | | Amount | % of |
| | | per Tablet | Tablet |
| Components | Function | (mg) | (w/w) |

ENTERIC COATED TABLET

| | | | |
|---|---|---|---|
| Famotidine USP/EP | Active | 20.0 | 21.2 |
| Anhydrous Lactose NF/EP | Filler | 13.1 | 13.9 |
| (Pharmatose DCL 21) | | | |
| Microcrystalline cellulose NF/EP | Thicking agent | 49.0 | 51.9 |
| (Avicel PH102) | | | |
| Croscarmellose sodium NF/EP | Filler | 6.0 | 6.3 |
| (Ac-Di-Sol) | | | |
| Colloidal silicon dioxide NF/EP | Binding agent | 0.5 | 0.5 |
| (Cab-O-Sil M5P) | | | |
| Magnesium Stearate NF | Lubricant | 1.4 | 1.5 |
| Methacrylic acid copolymer NF | Coating agent | 2.65 | 2.8 |
| (Eudragit L100-55) | | | |
| Talc USP/EP | Thicking agent | 0.53 | 0.6 |
| Triethyl Citrate NF/EP | Plasticizer | 1.32 | 1.4 |
| Purified Water USP/EP | Solvent | —¹ | 0.0 |
| Dehydrated Alcohol USP/EP | Solvent | —¹ | 0.0 |

| | | |
|---|---|---|
| Total Weight = | 94.5 | 100.0 |

FINAL TABLET

| | | | |
|---|---|---|---|
| Outer Tablet | | | |
| Naproxen Sodium USP/EP | Active | 550.0 | 48.9 |
| Famotidine USP/EP | Active | 20.0 | 1.8 |
| Microcrystalline cellulose NF/EP | Thicking agent | 165.0 | 14.7 |
| (Avicel PH102) | | | |
| Silicified microcrystalline cellulose | Binding agent | 165.0 | 14.7 |
| NF/EP (Prosolv SMCC 90) | | | |
| Povidone USP/EP | Filler | 50.0 | 4.4 |
| Croscarmellose sodium NF/EP | Filler | 30.0 | 2.7 |
| (Ac-Di-Sol) | | | |
| Magnesium Stearate NF | Lubricant | 40.0 | 3.6 |
| Colloidal silicone dioxide NF/EP | Binding agent | 10.0 | 0.9 |
| Inner Tablet | | | |
| Famotidine Tablet, 20 mg (EC) | In-process | 94.5 | 8.4 |
| | tablet | | |

| | | |
|---|---|---|
| Total Weight = | 1124.5 | 100.0 |

¹Water is removed during the process and therefore not factored in tablet weight.

Tablets prepared by the Applicants formulated as above were found not to have an acceptable delayed release of famotidine. It is believed that in the compression step the enteric coating was disrupted, allowing the famotidine to be released rapidly. It is therefore important that the compression settings be selected to avoid breaching the enteric coat.

### Example 3

### Immediate Release Blend of Naproxen Sodium and Famotidine

An immediate release blend of naproxen sodium and famotidine is prepared by blending naproxen sodium, famotidine, microcrystalline cellulose, and croscarmellose sodium in a v-blender.

The composition of the immediate release blend is shown in Table 4.

TABLE 4
Composition of immediate release formulation

| | | Amount per |
|---|---|---|
| Component | Function | capsule (mg) |
| Naproxen sodium | Active | 550 |
| Famotidine | Active | 20 |
| Microcrystalline cellulose | Filler | 150 |
| (Avicel PH102) | | |
| Croscarmellose sodium (AcDiSol) | Disintegrant | 30 |

### Example 4

### Immediate Release Blend of Naproxen Sodium and Famotidine

An immediate release blend of naproxen sodium and famotidine is prepared by blending naproxen sodium, famotidine, microcrystalline cellulose, povidone, croscarmellose sodium, colloidal silicon dioxide and magnesium stearate in a v-blender.

The composition of the immediate release blend is shown in Table 5.

TABLE 5
Composition of immediate release formulation

| | | Amount per |
|---|---|---|
| Component | Function | capsule (mg) |
| Naproxen sodium | Active | 550 |
| Famotidine | Active | 20 |
| Microcrystalline cellulose | Filler | 165 |
| (Avicel PH102) | | |
| Silicified microcrystalline | Filler | 165 |
| cellulose (Prosolv SMCC 90) | | |
| Povidone | Binder | 50 |
| Croscarmellose sodium (AcDiSol) | Disintegrant | 30 |
| Magnesium stearate | Lubricant | 40 |
| Colloidal silicon dioxide | Glidant | 10 |

### Example 5

### Delayed-Burst Release Osmotic Rupturing Multiparticulates

Osmotic rupturing multiparticulates are prepared in the following manner: an inert seed core comprised of microcrystalline cellulose is coated with the drug layer solution of 18% famotidine, 2% HPMC E5 premium, and 80% H2O in a fluid bed coater to a coating weight of 50 wt %. The drug layer coated beads are then coated with the sweller layer suspension of 18% croscarmellose sodium (AcDiSol), milled, 7% HPC (Klucel EF), and 75% ethanol in a fluid bed coater to a coating weight of 50 wt %. The sweller layer coated beads are then coated with the coating layer suspension of 10% ethylcellulose (Ethocel 10 cp), 10% talc, and 80% ethanol in a fluid bed coater to a coating weight of 10-50 wt %.

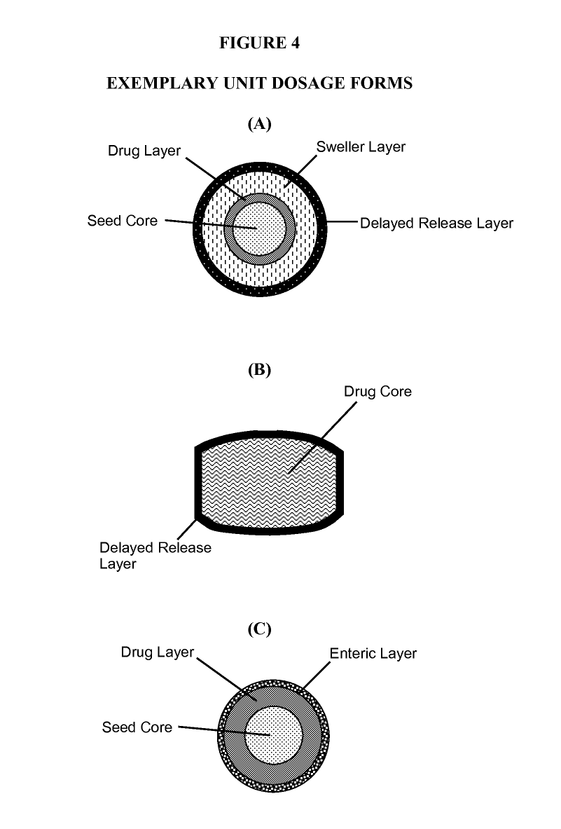

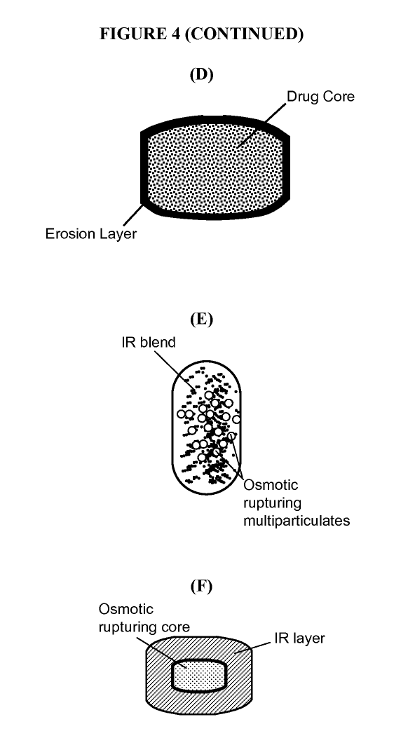

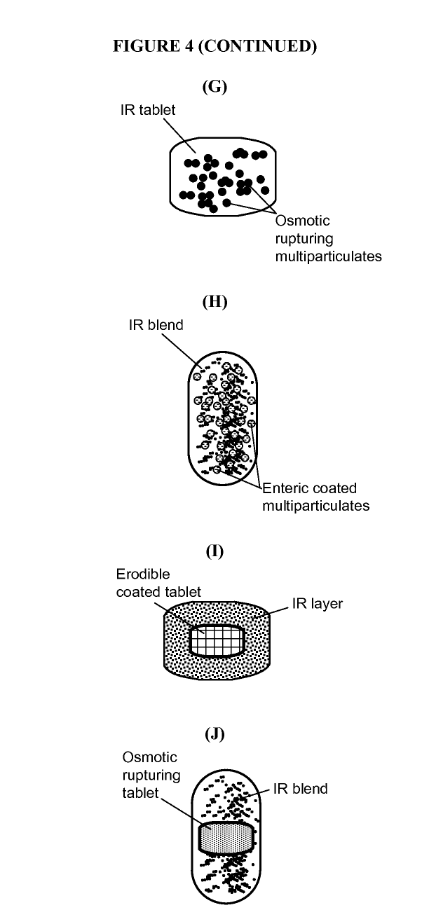

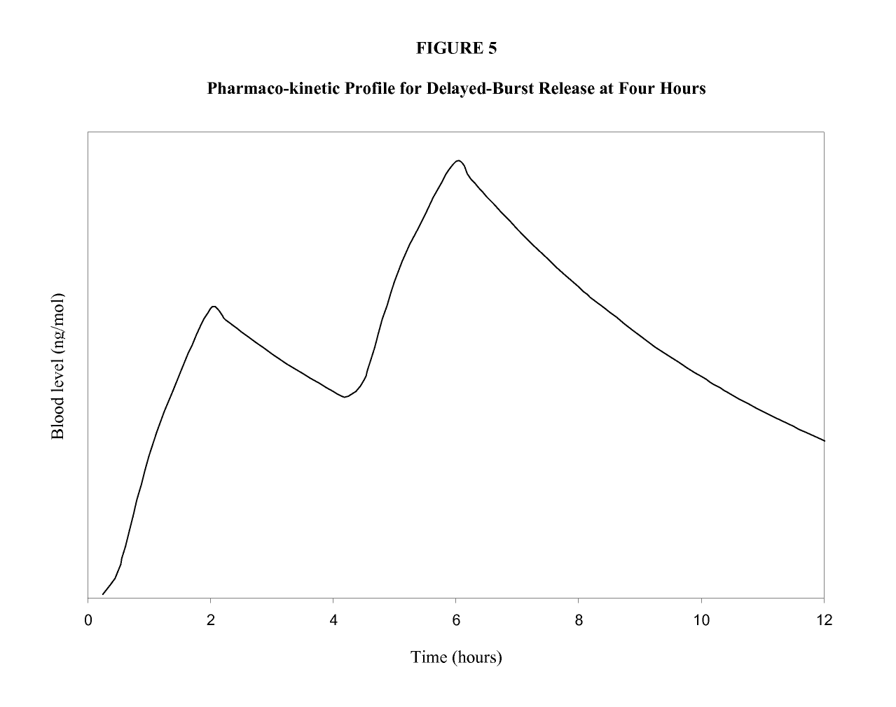

FIG. 4A shows a schematic of an osmotic rupturing multiparticulate.

The composition of the osmotic rupturing multiparticulates with a 20 wt % DR coating is shown in Table 6.

TABLE 6
Composition of osmotic rupturing multiparticulates

| | Amount (mg/g final | Component | | |
|---|---|---|---|---|
| Layer | multiparticulates) | (% in layer) | Name | Function |
| Seed | 200.0 | 500 μm | Microcrystalline | Substrate |
| Core | | Celphere | cellulose (Celphere | |
| | | | CP-506) | |
| Drug | 2.0 | HPMC (10%) | HPMC (E5 | Binder |
| layer | | | premium) | |
| | 180.0 | famotidine | API | Active |
| | | (90%) | | |
| Sweller | 114.3 | HPC (28.6%) | HPC (Klucel EF) | Binder |
| layer | 285.7 | Ac-Di-Sol | Croscarmellose | Sweller |
| | | (71.4%) | sodium (Ac-Di-Sol, | |
| | | | milled) | |
| Delayed | 100 | Talc (50%) | Talc (IMP-1889L) | Coating strength |
| release | | | | modifier |
| layer | 100 | Ethyl | Ethylcellulose | Semipermeable |
| | | cellulose | (Ethocel STD 10 cP) | coating |
| | | (50%) | | |

### Example 6

### Delayed-Burst Release Osmotic Rupturing Multiparticulates

Oosmotic rupturing multiparticulates are prepared in the following manner: an inert seed core comprised of microcrystalline cellulose is coated with a drug & sweller layer solution of 5% famotidine, 12% croscarmellose sodium, 3% HPC, and 80% ethanol in a fluid bed coater to a coating weight of 75 wt %. The drug layer coated beads are then coated with the coating layer suspension of 10% ethylcellulose, 10% talc, and 80% ethanol in a fluid bed coater to a coating weight of 20 wt %.

The composition of the osmotic rupturing multiparticulates is shown in Table 7.

TABLE 7
Composition of osmotic rupturing multiparticulates

| | Amount | | | |
|---|---|---|---|---|
| | (mg/g final | | | |
| | multi- | Component | | |
| Layer | particulates) | (% in layer) | Name | Function |
| Seed | 200.0 | | Microcrystalline | Substrate |
| Core | | | cellulose | |
| Drug | 360.0 | Ac-Di-Sol | Croscarmellose | Disintegrant/ |
| layer | | (60%) | sodium | Sweller |
| | 150.0 | famotidine | API | Active |
| | | (25%) | | |
| | 90.0 | HPC (15%) | HPC | Binder |
| Delayed | 100.0 | Talc (50%) | Talc | Coating |
| release | | | | modifier |
| layer | 100.0 | Ethyl | Ethylcellulose | Semipermeable |
| | | cellulose | | coating |
| | | (50%) | | |

### Example 7

### Delayed-Burst Release Osmotic Rupturing Multiparticulates

Multiparticulates are prepared in the following manner; a seed core is manufactured using pelletization technology for immediate release; comprised of 30% microcrystalline cellulose, 40% croscarmellose sodium, 20% famotidine and 10% povidone. These cores are then coated with a coating layer to a coating weight of 20 wt % (coat/total), containing a solids ratio of 1:1 ethylcellulose to talc at 20 wt % solids in ethanol.

The composition of the osmotic rupturing multiparticulates is shown in Table 8.

TABLE 8
Composition of osmotic rupturing multiparticulates

| | Amount | | | |
|---|---|---|---|---|
| | (mg/g final | | | |
| | multi- | Component | | |
| Layer | particulates) | (% in layer) | Name | Function |
| Seed | 240.0 | MCC (30%) | Microcrystalline | Pelletizing aid |
| Core | | | cellulose | |
| | 320.0 | Ac-Di-Sol | Croscarmellose | Disintegrant/ |
| | | (40%) | sodium | Sweller |
| | 160.0 | famotidine | API | Active |
| | | (20%) | | |
| | 80.0 | PVP (10%) | Povidone | Binder |
| Delayed | 100.0 | Talc (50%) | Talc | Coating |
| release | | | | modifier |
| layer | 100.0 | Ethyl | Ethylcellulose | Semipermeable |
| | | cellulose | | coating |
| | | (50%) | | |

### Example 8

### Delayed-Burst Release Osmotic Rupturing Multiparticulates

Multiparticulates are prepared in the following manner; a seed core is manufactured using pelletization technology for immediate release; comprised of 30% microcrystalline cellulose, 20% lactose, 20% mannitol, 20% famotidine and 10% povidone. These cores are then coated with a coating layer to a coating weight of 20 wt % (coat/total), containing a solids ratio of 1:1 ethylcellulose to talc at 20 wt % solids in ethanol.

The composition of the osmotic rupturing multiparticulates is shown in Table 9.

TABLE 9
Composition of osmotic rupturing multiparticulates

| | Amount | | | |
|---|---|---|---|---|
| | (mg/g final | | | |
| | multi- | Component | | |
| Layer | particulates) | (% in layer) | Name | Function |
| Seed | 240.0 | MCC (30%) | Microcrystalline | Pelletizing aid |
| Core | | | cellulose | |
| | 160.0 | Lactose | Lactose | Osmogen |
| | | (20%) | | |
| | 160.0 | Mannitol | Mannitol | Osmogen |
| | | (20%) | | |
| | 160.0 | famotidine | API | Active |
| | | (20%) | | |
| | 80.0 | PVP (10%) | Povidone | Binder |
| Delayed | 100.0 | Talc (50%) | Talc | Coating |
| release | | | | modifier |
| layer | 100.0 | Ethyl | Ethylcellulose | Semipermeable |
| | | cellulose | | coating |
| | | (50%) | | |

### Example 9

### Delayed-Burst Release Osmotic Rupturing Tablet

A tablet is prepared in the following manner; a blend of microcrystalline cellulose, famotidine, croscarmellose sodium, and magnesium stearate are prepared in a V-blender. The blend is compressed into a 100 mg tablets using ¼″ standard SRC tooling on a tablet press. These tablets are then coated with a coating layer to a coating weight of 10-50 wt % (coat/core), containing a solids ratio of 1:1 ethylcellulose to talc at 20 wt % solids in ethanol.

A schematic of an osmotic rupturing tablet is shown in FIG. 4B.

The composition of the osmotic rupturing tablet is shown in Table 10.

TABLE 10
Composition of osmotic rupturing tablet

| | Amount | Component | | |
|---|---|---|---|---|
| Layer | (mg) | (% in layer) | Name | Function |
| Tablet | 20.0 | famotidine | API | Active |
| | | (20%) | | |
| | 39.5 | MCC (39.5%) | Microcrystalline | Binder |
| | | | cellulose | |
| | 40.0 | Ac-Di-Sol | Croscarmellose | Disintegrant/ |
| | | (40%) | sodium | Sweller |
| | 0.5 | MgSt (0.5%) | Magnesium Stearate | Lubricant |
| Delayed | 15.0 | Talc (50%) | Talc | Coating |
| release | | | | modifier |
| layer | 15.0 | Ethyl | Ethylcellulose | Semipermeable |
| | | cellulose | | coating |
| | | (50%) | | |

### Example 10

### Delayed-Burst Enteric Coated Multiparticulate

The enteric coated multiparticulates are prepared in the following manner: an inert seed core comprised of microcrystalline cellulose is coated with a drug layer solution of 15% famotidine, 5% HPMC, and 80% water in a fluid bed coater to a coating weight of 25 wt %. The drug layer coated beads are then coated with the enteric coating layer composed of 14% methacrylic acid copolymer, 4% triethyl citrate, 2% talc, and 80% ethanol (95%) in a fluid bed coater to a coating weight of 5-30 wt %.

A schematic of an enteric coated multiparticulate is shown in FIG. 4(C).

The composition of the enteric coated multiparticulate is shown in Table 11.

TABLE 11
Composition of enteric coated multiparticulate

| | Amount | | | |
|---|---|---|---|---|
| | (mg/g final | | | |
| | multi- | Component | | |
| Layer | particulates) | (% in layer) | Name | Function |
| Seed | 600.0 | (100%) | Microcrystalline | Substrate |
| Core | | | cellulose | |
| Drug | 150.0 | famotidine | API | Active |
| layer | | (75%) | | |
| | 50.0 | HPMC (25%) | HPMC | Binder |
| Enteric | 140.0 | Eudragit | Methyacrylic acid | Coating |
| layer | | L100 (70%) | copolymer, Type A | agent |
| | 40.0 | TEC (20%) | Triethyl citrate | Plasticizer |
| | 20.0 | Talc (10%) | Talc | Coating |
| | | | | modifier |

### Example 11

### Delayed-Burst Enteric Coated Multiparticulate

The multiparticulates are prepared in the following manner; a seed core is manufactured using pelletization technology for immediate release; comprised of 50% microcrystalline cellulose, 10% lactose, 10% croscarmellose sodium, 20% famotidine and 10% povidone. These cores are then coated beads are then coated with the enteric coating layer composed of 14% methacrylic acid copolymer, 4% triethyl citrate, 2% talc, and 80% ethanol (95%) in a fluid bed coater to a coating weight of 10 wt %.

The composition of the enteric coated multiparticulate is shown in Table 12.

TABLE 12
Composition of enteric coated multiparticulate

| | Amount | | | |
|---|---|---|---|---|
| | (mg/g final | | | |
| | multi- | Component | | |
| Layer | particulates) | (% in layer) | Name | Function |
| Seed | 450.0 | MCC (50%) | Microcrystalline | Pelletizing |
| Core | | | cellulose | aid |
| | 90.0 | Lactose | Lactose | Filler |
| | | (10%) | | |
| | 90.0 | AcDiSol | Mannitol | Disintegrant |
| | | (10%) | | |
| | 180.0 | famotidine | API | Active |
| | | (20%) | | |
| | 90.0 | PVP (10%) | Povidone | Binder |
| Enteric | 70.0 | Eudragit | Methyacrylic acid | Coating |
| layer | | L100 (70%) | copolymer, Type A | agent |
| | 20.0 | TEC (20%) | Triethyl citrate | Plasticizer |
| | 10.0 | Talc (10%) | Talc | Coating |
| | | | | modifier |

### Example 12

### Delayed-Burst Enteric Coated Tablet

A tablet is prepared in the following manner; a blend of microcrystalline cellulose, lactose, famotidine, croscarmellose sodium, colloidal silicon dioxide, and magnesium stearate are prepared in a V-blender. The blend is compressed into a 100 mg tablets using ¼″ standard SRC tooling on a tablet press. These tablets are then coated with the enteric coating layer composed of suspension of 14% methacrylic acid copolymer, 4% triethyl citrate, 2% talc, and 80% ethanol (95%) in a fluid bed or pan coater to a coating weight of 10 wt % (coat/tablet).

The composition of the enteric coated tablet is shown in Table 13.

TABLE 13
Composition of enteric coated tablet

| | Amount | Component | | |
|---|---|---|---|---|
| Layer | (mg) | (% in layer) | Name | Function |
| Tablet | 20.0 | famotidine | API | Active |
| | | (20%) | | |
| | 15.0 | Lactose | Lactose | Filler |
| | | (15%) | | |
| | 50.0 | MCC (50%) | Microcrystalline | Binder |
| | | | cellulose | |
| | 10.0 | Ac-Di-Sol | Croscarmellose | Disintegrant |
| | | (10%) | sodium | |
| | 4.0 | Cab-O-Sil | Colloidal silicon | Flow aid |
| | | (4%) | dioxide | |
| | 1.0 | MgSt (1%) | Magnesium Stearate | Lubricant |
| Enteric | 7.0 | Eudragit | Methyacrylic acid | Coating agent |
| layer | | L100 (70%) | copolymer, Type A | |
| | 2.0 | TEC (20%) | Triethyl citrate | Plasticizer |
| | 1.0 | Talc (10%) | Talc | Coating modifier |

### Example 13

### Delayed-Burst Erodible Coated Tablet

A tablet is prepared in the following manner; a blend of microcrystalline cellulose, lactose, famotidine, croscarmellose sodium, colloidal silicon dioxide, and magnesium stearate are prepared in a V-blender. The blend is compressed into a 100 mg tablets using ¼″ standard SRC tooling on a tablet press. These tablets are then coated with an erosion coating layer composed of 4.5% high viscosity grade HPMC, 0.5% PEG 400 in 95% water in a fluid bed or pan coater to a coating weight of 10-50 wt % (coat/tablet).

A schematic of an erodible coated tablet is shown in FIG. 4D. The composition of the erodible coated tablet is shown in Table 14.

TABLE 14
Composition of erodible coated tablet

| | Amount | Component | | |
|---|---|---|---|---|
| Layer | (mg) | (% in layer) | Name | Function |
| Tablet | 20.0 | famotidine | API | Active |
| | | (20%) | | |
| | 15.0 | Lactose | Lactose | Filler |
| | | (15%) | | |
| | 50.0 | MCC (50%) | Microcrystalline | Binder |
| | | | cellulose | |
| | 10.0 | Ac-Di-Sol | Croscarmellose | Disintegrant |
| | | (10%) | sodium | |
| | 4.0 | Cab-O-Sil | Colloidal silicon | Flow aid |
| | | (4%) | dioxide | |
| | 1.0 | MgSt (1%) | Magnesium Stearate | Lubricant |
| Erosion | 45.0 | HPMC (90%) | HPMC (Methocel | Coating agent |
| layer | | | K4M) | |
| | 5.0 | PEG 400 | PEG 400 | Plasticizer |
| | | (10%) | | |

### Example 14

### Osmotic Rupturing Multiparticulates with an Immediate Release Blend in a Capsule

An exemplary unit dosage form in accordance with certain embodiments of the invention can be prepared as follows. In this example, the delayed-burst release formulation is in the form of osmotic rupturing famotidine multiparticulates (Example 5), while the immediate release formulation of famotidine and naproxen is prepared as a flowable powder (Example 3).

To assemble the final unit dose formulation, 111 mg of the osmotic rupturing multiparticulates (Example 5) and 750 mg of the immediate release blend (Example 3) are filled into a hard gelatin capsule shell on a capsule filling machine.

A schematic of this dosage form is shown in FIG. 4E.

### Example 15

### Osmotic Rupturing Multiparticulates with an Immediate Release Blend in a Capsule

An exemplary unit dosage form in accordance with certain embodiments of the invention can be prepared as follows. In this example, the delayed-burst release formulation is in the form of osmotic rupturing famotidine multiparticulates (Example 6), while the immediate release formulation of famotidine and naproxen is prepared as a flowable powder (Example 3).

To assemble the final unit dose formulation, 133 mg of the osmotic rupturing multiparticulates (Example 6) and 750 mg of the immediate release blend (Example 3) are filled into a hard gelatin capsule shell on a capsule filling machine.

### Example 16

### Osmotic Rupturing Multiparticulates with an Immediate Release Blend in a Capsule

An exemplary unit dosage form in accordance with certain embodiments of the invention can be prepared as follows. In this example, the delayed-burst release formulation is in the form of osmotic rupturing famotidine multiparticulates (Example 7), while the immediate release formulation of famotidine and naproxen is prepared as a flowable powder (Example 3).

To assemble the final unit dose formulation, 125 mg of the osmotic rupturing multiparticulates (Example 7) and 750 mg of the immediate release blend (Example 3) are filled into a hard gelatin capsule shell on a capsule filling machine.

### Example 17

### Osmotic Rupturing Multiparticulates with an Immediate Release Blend in a Capsule

An exemplary unit dosage form in accordance with certain embodiments of the invention can be prepared as follows. In this example, the delayed-burst release formulation is in the form of osmotic rupturing famotidine multiparticulates (Example 8), while the immediate release formulation of famotidine and naproxen is prepared as a flowable powder (Example 3).

To assemble the final unit dose formulation, 125 mg of the osmotic rupturing multiparticulates (Example 8) and 750 mg of the immediate release blend (Example 3) are filled into a hard gelatin capsule shell on a capsule filling machine.

### Example 18

### Osmotic Rupturing Tablet in an Immediate Release Tablet

An exemplary unit dosage form in accordance with certain embodiments of the invention can be prepared as follows. In this example, the delayed-burst release formulation is in the form of osmotic rupturing famotidine tablet (Example 9), while the immediate release formulation of famotidine and naproxen is prepared as a flowable powder (Example 4).

To assemble the final unit dose formulation, one osmotic rupturing tablet (Example 9) and 1030 mg of the immediate release blend (Example 4) are compressed into a tablet within a tablet configuration using a tablet press.

A schematic of this dosage form is shown in FIG. 4F.

### Example 19

### Osmotic Rupturing Multiparticulates in an Immediate Release Tablet

An exemplary unit dosage form in accordance with certain embodiments of the invention can be prepared as follows. In this example, the delayed-burst release formulation is in the form of osmotic rupturing famotidine multiparticulates (Example 5), while the immediate release formulation of famotidine and naproxen is prepared as a flowable powder (Example 4).

To assemble the final unit dose formulation, 111 mg of the osmotic rupturing multiparticulates (Example 5) and 1030 mg of the immediate release blend (Example 4) are compressed into a tablet using a tablet press.

A schematic of this dosage form is shown in FIG. 4G.

### Example 20

### Enteric Coated Multiparticulates with an Immediate Release Blend in a Capsule

An exemplary unit dosage form in accordance with certain embodiments of the invention can be prepared as follows. In this example, the delayed-burst release formulation is in the form of enteric coated famotidine multiparticulates (Example 10), while the immediate release formulation of famotidine and naproxen is prepared as a flowable powder (Example 3).

To assemble the final unit dose formulation, 133 mg of the enteric coated multiparticulates (Example 10) and 750 mg of the immediate release blend (Example 3) are filled into a hard gelatin capsule shell on a capsule filling machine.

A schematic of this dosage form is shown in FIG. 4H.

### Example 21

### Enteric Coated Multiparticulates with an Immediate Release Blend in a Capsule

An exemplary unit dosage form in accordance with certain embodiments of the invention can be prepared as follows. In this example, the delayed-burst release formulation is in the form of enteric coated famotidine multiparticulates (Example 11), while the immediate release formulation of famotidine and naproxen is prepared as a flowable powder (Example 3).

To assemble the final unit dose formulation, 111 mg of the enteric coated multiparticulates (Example 11) and 750 mg of the immediate release blend (Example 3) are filled into a hard gelatin capsule shell on a capsule filling machine.

### Example 22

### Enteric Coated Tablet in an Immediate Release Tablet

An exemplary unit dosage form in accordance with certain embodiments of the invention can be prepared as follows. In this example, the delayed-burst release formulation is in the form of enteric coated famotidine tablet (Example 12), while the immediate release formulation of famotidine and naproxen is prepared as a flowable powder (Example 4).

To assemble the final unit dose formulation, one enteric coated tablet (Example 12) and 1030 mg of the immediate release blend (Example 4) are compressed into a tablet within a tablet configuration using a tablet press.

### Example 23

### Erodible Coated Tablet in an Immediate Release Tablet

An exemplary unit dosage form in accordance with certain embodiments of the invention can be prepared as follows. In this example, the delayed-burst release formulation is in the form of erodible coated famotidine tablet (Example 13), while the immediate release formulation of famotidine and naproxen is prepared as a flowable powder (Example 4).

To assemble the final unit dose formulation, one erodible coated tablet (Example 13) and 1030 mg of the immediate release blend (Example 4) are compressed into a tablet within a tablet configuration using a tablet press.

A schematic of this dosage form is shown in FIG. 4I.

### Example 24

### Osmotic Rupturing Tablet with an Immediate Release Blend in a Capsule

An exemplary unit dosage form in accordance with certain embodiments of the invention can be prepared as follows. In this example, the delayed-burst release formulation is in the form of osmotic rupturing famotidine tablet (Example 9), while the immediate release formulation of famotidine and naproxen is prepared as a flowable powder (Example 3).

To assemble the final unit dose formulation, one osmotic rupturing tablet (Example 9) and 750 mg of the immediate release blend (Example 3) are filled into a hard gelatin capsule shell on a capsule filling machine.

A schematic of this dosage form is shown in FIG. 4J.

All references (including literature and patent applications) cited above are hereby expressly incorporated by reference in their entirety for all purposes.

1. A unit dosage form, comprising:
(a) an immediate-release famotidine formulation;
(b) an immediate-release naproxen formulation; and
(c) a delayed-burst release famotidine formulation comprising famotidine and a release-delaying agent.

2. The unit dosage form of claim 1, wherein:
(a) the immediate-release famotidine formulation comprises from about 10 mg to about 30 mg famotidine;
(b) the immediate-release naproxen formulation comprises from about 200 mg to about 600 mg naproxen; and
(c) the delayed-burst release famotidine formulation comprises from about 10 mg to about 80 mg of famotidine, and a release-delaying agent.

3. The unit dosage form of claim 2, wherein the amount of famotidine in the delayed-burst release famotidine formulation is about the same as the amount of famotidine in the immediate release famotidine formulation.

4. The unit dosage form of claim 2, wherein the delayed-burst release famotidine formulation and the immediate release famotidine formulation each comprise about 20 mgs famotidine.

5. The unit dosage form of claim 2, wherein the amount of famotidine in the delayed-burst release famotidine formulation is greater than then amount of famotidine in the immediate release famotidine formulation.

6. The unit dosage form of claim 4, wherein the amount of famotidine in the delayed-burst release famotidine formulation is about 1.2-fold to 4-fold greater than the amount of famotidine in the immediate release famotidine formulation.

7. The unit dosage form of claim 4, wherein the unit dosage form contains about 200-600 mg naproxen, about 10-30 mg famotidine in the immediate release famotidine formulation, and about 20-80 mg famotidine in the delayed-burst release famotidine formulation.

8. The unit dosage form of claim 1, wherein the delayed-burst release famotidine formulation has a burst duration of about 60 minutes or less.

9. The unit dosage form of claim 7, wherein the delayed-burst release famotidine formulation has a burst duration of about 30 minutes or less.

10. The unit dosage form of claim 1, wherein release from (a) and/or (b) occurs within about 30 minutes after administration.

11. The unit dosage form of claim 1, wherein the release interval between (a) and (c) is at least about 1 hour.

12. The unit dosage form of claim 10, wherein the release is about 4 hours.

13. The unit dosage form of claim 1, wherein the famotidine from (a) and/or (c) is effective to raise gastric pH above about 4.5 for at least 4 hours.

14. The unit dosage form of claim 1, wherein the delayed-burst release of the famotidine from (c) extends the period during which gastric pH is raised.

15. The unit dosage form of claim 12, wherein the gastric pH is raised to a level that remains below about pH 6.5.

16. The unit dosage form of claim 1, wherein the release-delaying agent is formulated for a fixed-time delayed release.

17. The unit dosage form of claim 15, wherein the delayed-burst release famotidine formulation releases famotidine through osmotic rupture.

18. The unit dosage form of claim 15, wherein the delayed-burst release famotidine formulation begins to release famotidine at least about 4 hours after administration to a subject.

19. The unit dosage form of claim 1, wherein the release-delaying agent comprises an enteric material.

20. The unit dosage form of claim 18, wherein the delayed-burst release famotidine formulation releases famotidine at or above a pH of about 6.0.

21. The unit dosage form of claim 19, wherein the famotidine from (a) raises gastric pH above about 3.5 and below about 6.0.

22. The unit dosage form of claim 18, wherein the enteric material allows release at or above a pH of about 5.5.

23. The unit dosage form of claim 21, wherein the famotidine from (a) raises gastric pH above about 3.5 and below about 5.5.

24. The unit dosage form of claim 18, wherein said enteric barrier comprises a material selected from the group consisting of cellulosic polymers, methacrylates, vinyl polymers and copolymers, and combinations thereof.

25. The unit dosage form of claim 1, wherein (a), (b) and/or (c) comprises one or more pharmaceutically acceptable excipients selected from the group consisting of sugars, soluble salts, colorants, fillers, disintegrants, glidants, anti-lacking agents, anti-static agents, and combinations thereof.

26. The unit dosage form of claim 1, for treating or preventing pain or inflammation in a subject.

27. The unit dosage form of claim 1, wherein the unit dosage form is administered twice a day.

28. The unit dosage form of claim 1, for reducing the risk of an adverse side-effect of an NSAID.

29. A method for treating or preventing pain or inflammation in a subject, comprising administering a unit dosage form of claim 1.

30. The method of claim 29, wherein the unit dosage form is administered twice a day.

31. A method for manufacture of a unit dosage form, comprising
(a) preparing an immediate release formulation comprising about 200-600 mg naproxen;
(b) preparing an immediate release formulation comprising about 10-30 mg famotidine;
(c) preparing a delayed-burst release formulation comprising about 40-80 mg famotidine; and
(d) combining the formulations of steps (a), (b) and (c), thereby resulting in a unit dosage form.
